(19) United States
(12) Patent Application Publication
(10) Pub. No.: US 2019/0297818 A1
(43) Pub. Date: Oct. 3, 2019

(54) ARTICHOKE VARIETIES NAMED 'PAGA G-1', 'PAGA 13-1', AND 'PAGA 15-1'

(71) Applicant: Michael R. SCATTINI, Salinas, CA (US)

(72) Inventor: Michael R. SCATTINI, Salinas, CA (US)

(21) Appl. No.: 16/370,551

(22) Filed: Mar. 29, 2019

Related U.S. Application Data
(60) Provisional application No. 62/650,945, filed on Mar. 30, 2018.
(60) Provisional application No. 62/650,952, filed on Mar. 30, 2018.
(60) Provisional application No. 62/650,988, filed on Mar. 30, 2018.

(51) Int. Cl. A01H 6/14; A01H 5/02

(52) U.S. Cl. CPC A01H 6/1432; A01H 5/02

(57) ABSTRACT

New varieties of artichoke (Cynara cardunculus var. scolymus L.) plant named ‘PAGA G-1’, ‘PAGA 13-1’, and ‘PAGA 15-1’, particularly characterized by high yield, large primary bud size, large number of secondary and tertiary buds, and improved uniformity, are disclosed.

### CROSS REFERENCE TO RELATED APPLICATIONS

This application claims the benefits of U.S. Prov. App. Nos. 62/650,988, 62/650,952, and 62/650,945, all of which were filed Mar. 30, 2018 and are hereby incorporated by reference in their entirety.

### FIELD OF THE INVENTION

The present invention relates to the field of plant breeding. In particular, this invention relates to new varieties of artichoke (Cynara cardunculus L. var. scolymus) plant designated ‘PAGA G-1’, ‘PAGA 13-1’, and ‘PAGA 15-1’.

### BACKGROUND OF THE INVENTION

Artichoke (Cynara cardunculus L. var. scolymus), or globe artichoke, is a thistle-like perennial plant native to the Mediterranean region and is a member of the family Asteraceae. Artichoke belongs to the species Cynara cardunculus L. that includes two subspecies/cultivation groups: the cardoon (Cynara cardunculus L. var. altilis) that is cultivated for its edible leaf stems, and the artichoke that is cultivated for its edible immature inflorescence hereinafter referred to as buds, heads, or capitula. In some instances, artichoke is distinguished as Cynara scolymus.

Artichoke is a popular vegetable food source in many regions of the world, with nearly two million tons of artichokes produced annually. The United States produces approximately 40,000 tons of artichokes annually, with California being responsible for nearly 100% of its production, of which about 80% is grown in Monterey County.

Historically, a common perennial artichoke variety that is grown in the United States is ‘Green Globe’, also referred to as an “Heirloom” artichoke. Despite the use of it as a traditional cultivar, ‘Green Globe’ does not produce large numbers of large buds which are most desired by consumers. Further, ‘Green Globe’ exhibits a substantial amount of phenotypic variation as well as protracted and unpredictable harvest intervals.

Thus, there exists a need for improved artichoke varieties having large sized post-primary bud production, programmable and predictable harvest timing, and better uniformity.

### BRIEF SUMMARY OF THE INVENTION

In order to meet the above and other needs, the present invention is directed to improved artichoke plant varieties.

In some embodiments, the present invention is directed to an artichoke plant variety designated ‘PAGA G-1’, wherein a representative sample of tissue culture of said variety has been deposited under DSMZ Accession Number X1. In some embodiments, the present invention is directed to an artichoke plant variety designated ‘PAGA 13-1’, wherein a representative sample of tissue culture of said variety has been deposited under DSMZ Accession Number X2. In some embodiments, the present invention is directed to an artichoke plant variety designated ‘PAGA 15-1’, wherein a representative sample of tissue culture of said variety has been deposited under DSMZ Accession Number X3.

In some embodiments, the present invention is directed to an artichoke plant regenerated from the tissue culture of ‘PAGA G-1’, ‘PAGA 13-1’, or ‘PAGA 15-1’, wherein said plant expressing all the morphological and physiological characteristics of ‘PAGA G-1’, ‘PAGA 13-1’, or ‘PAGA 15-1’.

In some embodiments, the present invention is directed to a plant part from the plant of ‘PAGA G-1’, ‘PAGA 13-1’, or ‘PAGA 15-1’. In some embodiments, the plant part is a capitulum, a flower, a leaf, a stem, a seed, a cell, a tissue culture, or a portion thereof.

In some embodiments, the present invention is directed to a method of producing an artichoke plant, comprising crossing the plant of ‘PAGA G-1’, ‘PAGA 13-1’, or ‘PAGA 15-1’ with a second artichoke plant one or more times, and selecting progeny from said crossing.

In some embodiments, the present invention is directed to a method of producing an artichoke plant, comprising selfing the plant of ‘PAGA G-1’, ‘PAGA 13-1’, or ‘PAGA 15-1’ one or more times, and selecting progeny from said selfing.

In some embodiments, the present invention is directed to progeny of the artichoke plant variety of ‘PAGA G-1’, ‘PAGA 13-1’, or ‘PAGA 15-1’, wherein said progeny retain all the physiological and morphological characteristics of variety ‘PAGA G-1’, ‘PAGA 13-1’, or ‘PAGA 15-1’ when grown under the same environmental conditions.

In some embodiments, the present invention is directed to an essentially derived variety (EDV) of the artichoke plant variety ‘PAGA G-1’, ‘PAGA 13-1’, or ‘PAGA 15-1’ having one, two or three physiological and/or morphological characteristics that are different from those of the plant of ‘PAGA G-1’, ‘PAGA 13-1’, or ‘PAGA 15-1’, but otherwise having all the physiological and morphological characteristics of ‘PAGA G-1’, ‘PAGA 13-1’, or ‘PAGA 15-1’.

In some embodiments, the present invention is directed to a method of producing an artichoke plant, or a part thereof, of the artichoke plant variety ‘PAGA G-1’, ‘PAGA 13-1’, or ‘PAGA 15-1’, comprising vegetative propagation of the plant of ‘PAGA G-1’, ‘PAGA 13-1’, or ‘PAGA 15-1’. In some embodiments, the vegetative propagation comprises regenerating a whole artichoke plant from a part of ‘PAGA G-1’, ‘PAGA 13-1’, or ‘PAGA 15-1’. In some embodiments, the part is a cutting, a cell culture, a tissue culture, a stump, a sucker, a shoot, an offshoot or an ovoli. In some embodiments, the regenerated whole artichoke plant has all the morphological and physiological characteristics of ‘PAGA G-1’, ‘PAGA 13-1’, or ‘PAGA 15-1’ when grown under the same environmental conditions. In some embodiments, the present invention is directed to a plant part of the regenerated whole artichoke plant, wherein said plant part is a harvested capitulum, a pollen grain, a cell, a leaf, a petiole, a shoot, a stem, a root, a cutting, a stump, an offshoot, an ovoli, a receptacle, a bract, a flower, a floret, a flower bud, a seed, or a portion thereof. In some embodiments, the present invention is directed to a food or feed product comprising said plant part. In some embodiments, the plant part is fresh. In some embodiments, the plant part is processed.

In some embodiments, the present invention is directed to a method of producing an artichoke plant having a desired trait, wherein the method comprises transforming the artichoke plant of ‘PAGA G-1’, ‘PAGA 13-1’, or ‘PAGA 15-1’ with a transgene that confers the desired trait, wherein the transformed plant retains all the phenotypic and morphological characteristics of variety ‘PAGA G-1’, ‘PAGA 13-1’, or ‘PAGA 15-1’ and contains the desired trait.

In some embodiments, the present invention is directed to a method of determining the genotype of the plant of ‘PAGA G-1’, ‘PAGA 13-1’, or ‘PAGA 15-1’ comprising obtaining a sample of nucleic acids from said plant and detecting in said nucleic acids a plurality of polymorphisms.

### DESCRIPTION OF THE DRAWINGS

The patent or application file contains at least one drawing executed in color. Copies of this patent or patent application publication with color drawings will be provided by the Office upon request and payment of the necessary fee.

The accompanying photographs illustrate the overall appearances of the new artichoke plant varieties ‘PAGA G-1’, ‘PAGA 13-1’, and ‘PAGA 15-1’ showing the colors as true as is reasonably possible with colored reproductions of this type. Colors in the photographs may differ slightly from the color values cited in the detailed botanical description, which accurately describes the color of this variety. The depicted plant and plant parts of the new artichoke plant varieties ‘PAGA G-1’, ‘PAGA 13-1’, and ‘PAGA 15-1’ are from plants that are 5-6 months old, grown in Castroville, Calif.

FIG. 1 shows a large sized capitulum (bud) of ‘PAGA G-1’.

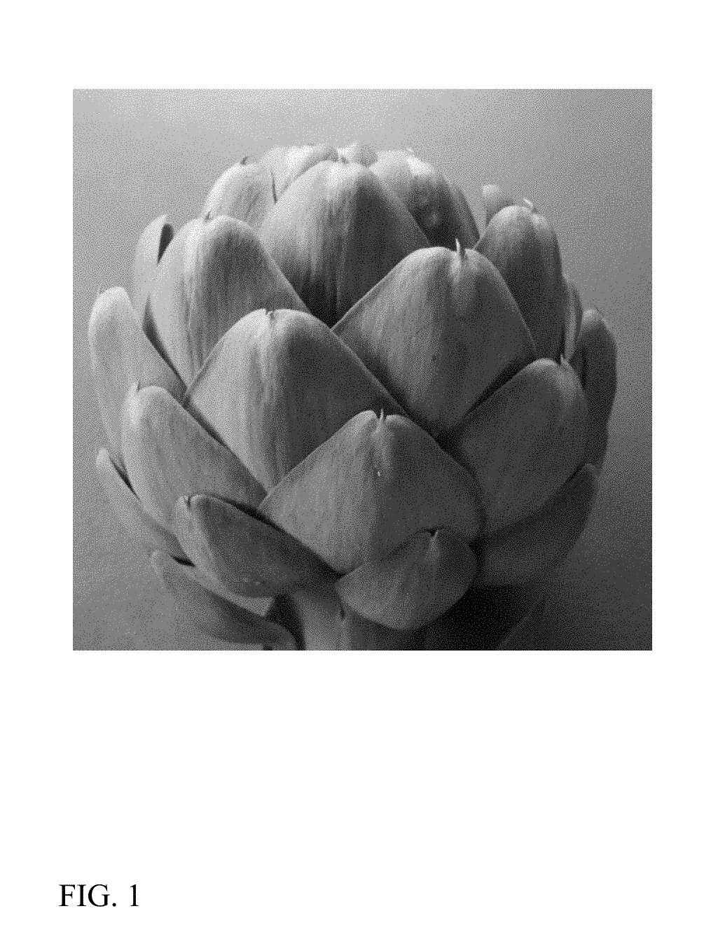

FIG. 2 shows an interior view of a bract of ‘PAGA G-1’.

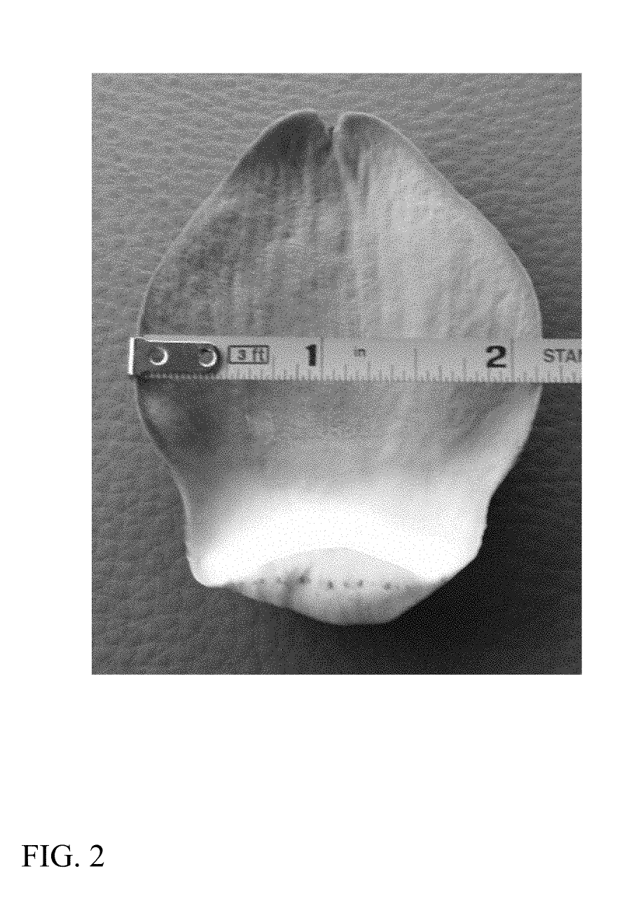

FIG. 3 shows an exterior view of a bract of ‘PAGA G-1’.

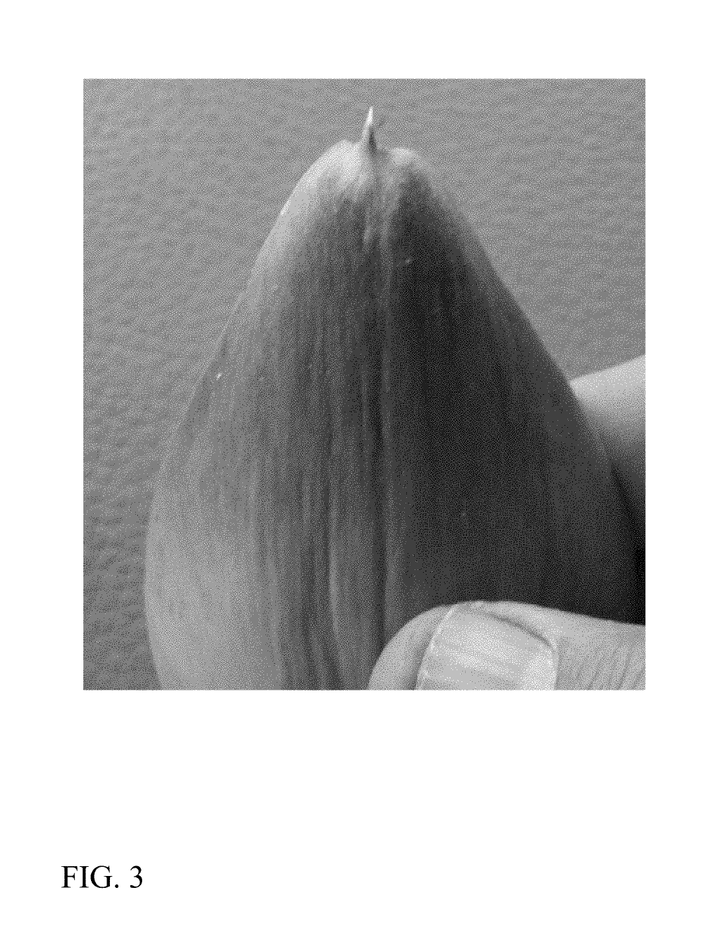

FIG. 4 shows a cross section of a capitulum (bud) of “PAGA G-1’.

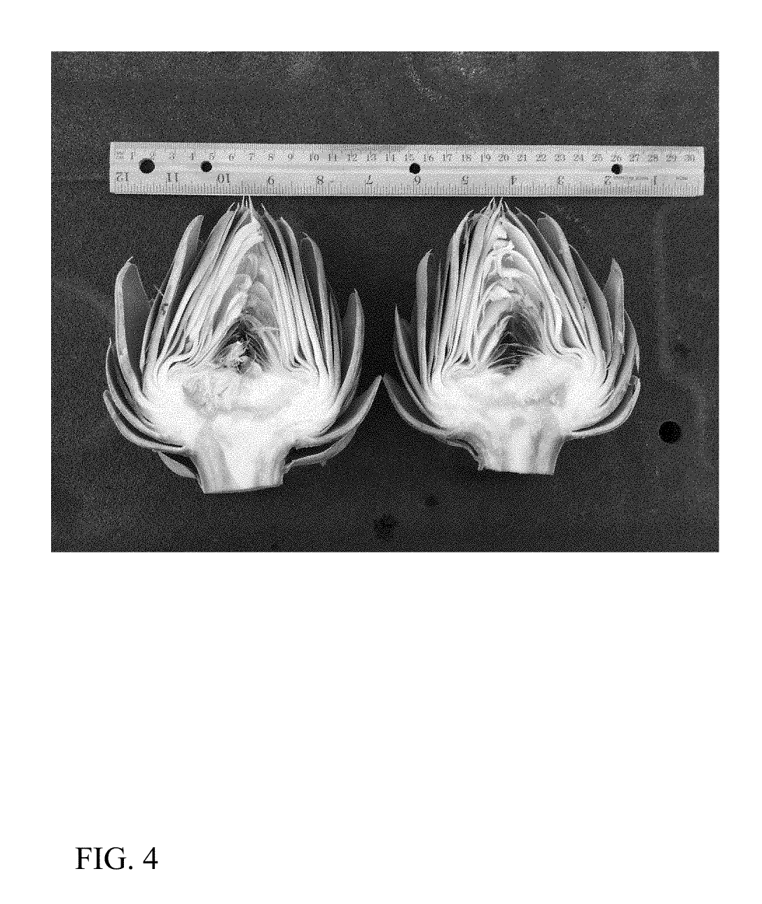

FIG. 5 shows various sized capitula (buds) of a ‘PAGA G-1’ plant.

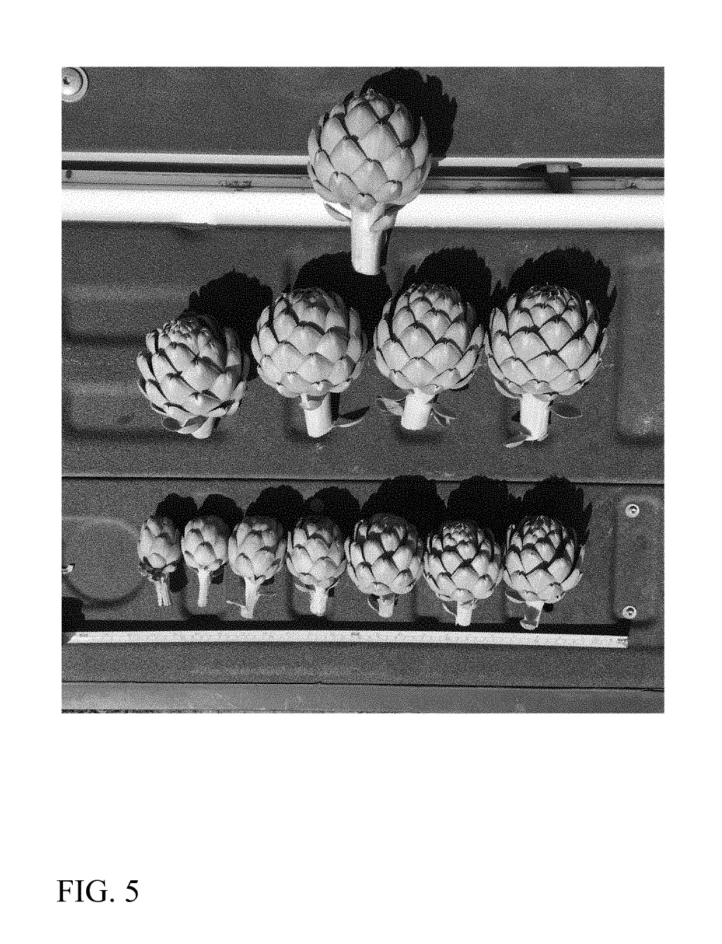

FIG. 6 shows a basal rosette leaf of ‘PAGA G-1’.

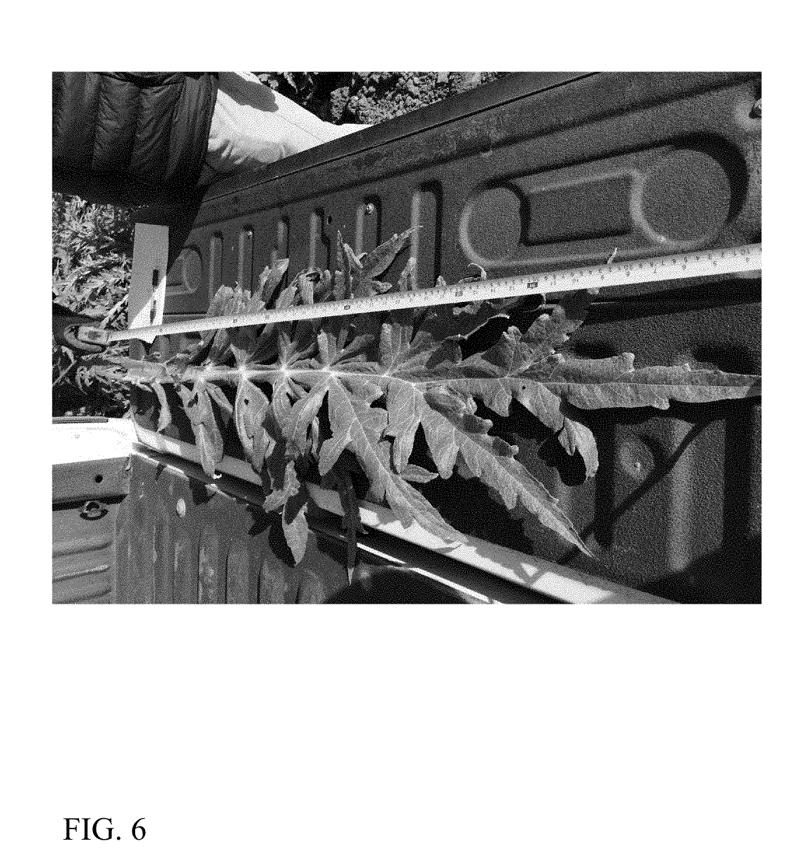

FIG. 7 shows a growing field of ‘PAGA G-1’ demonstrating its uniformity.

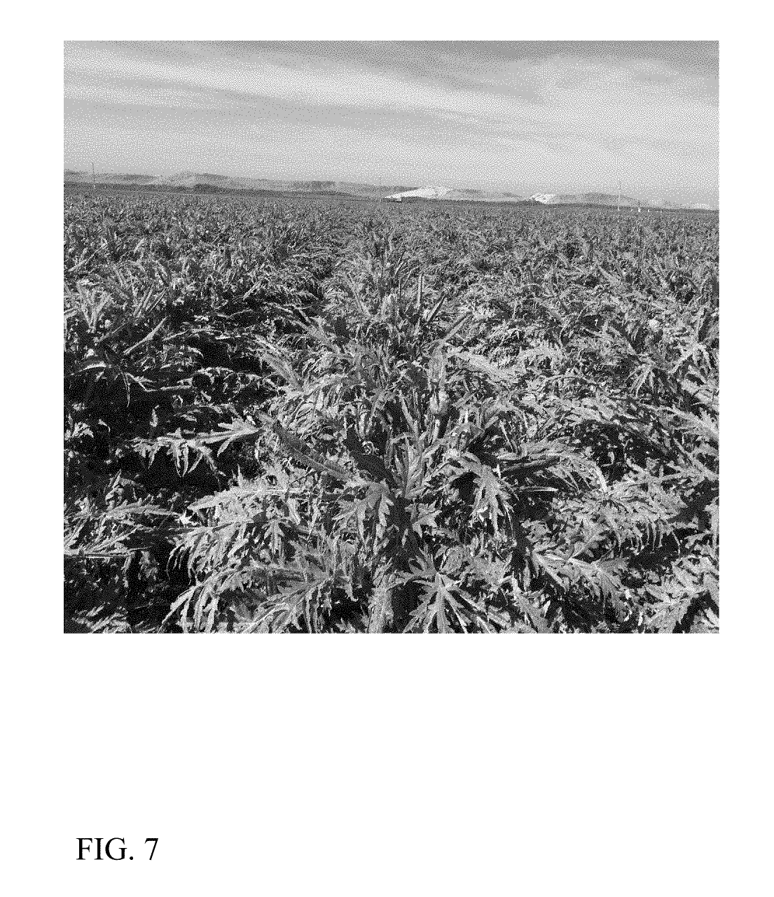

FIG. 8 shows a growing field of the parent variety ‘Green Globe’ demonstrating its lack of uniformity.

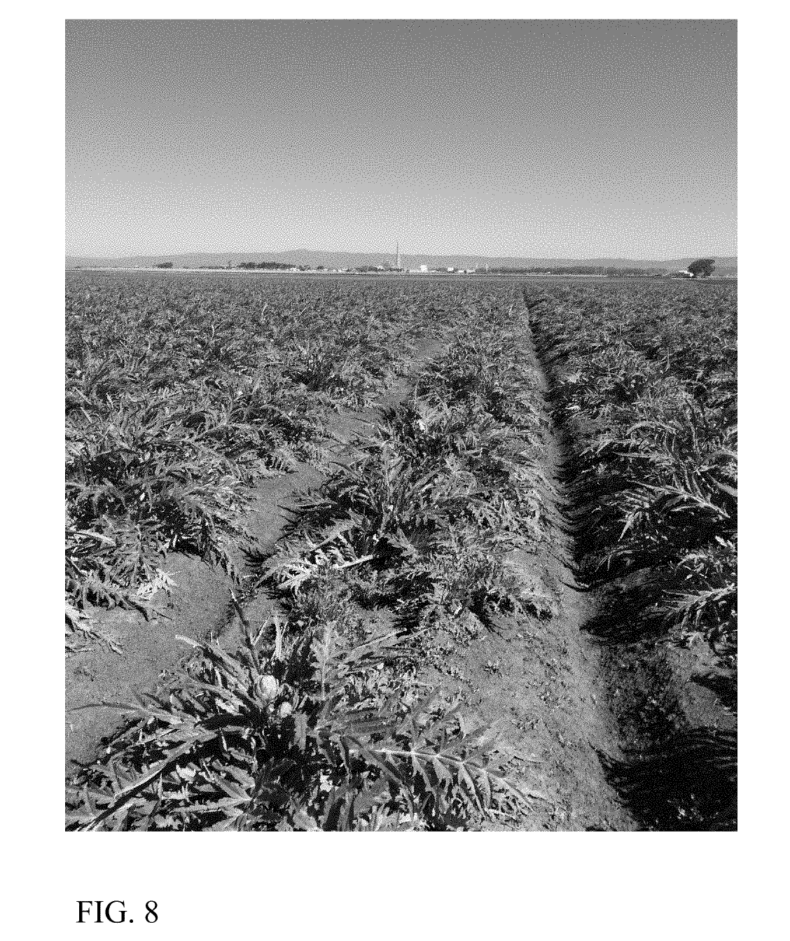

FIG. 9 shows ‘PAGA G-1’ (right) and ‘Green Globe’ (left) capitulum (bud) bases showing the differences in purple colored striations on lower bracts.

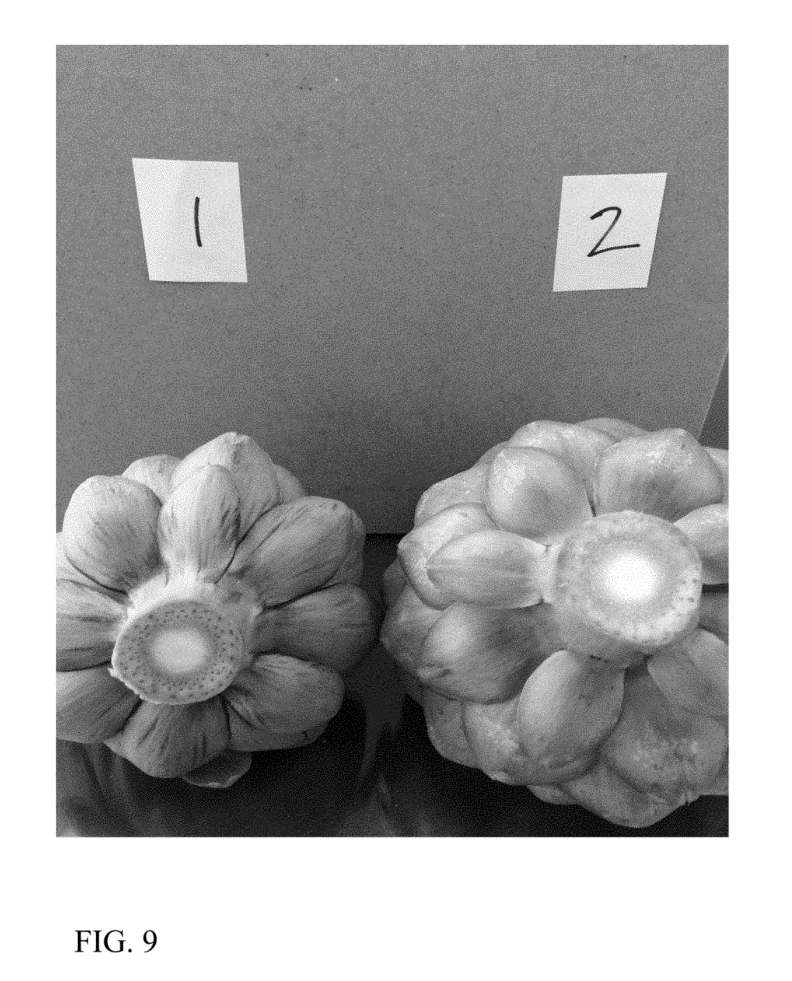

FIG. 10 shows ‘PAGA G-1’ (left) and ‘Green Globe’ (right) reproductive stems that have been stripped of leaves. The plants are both approximately 5 months old.

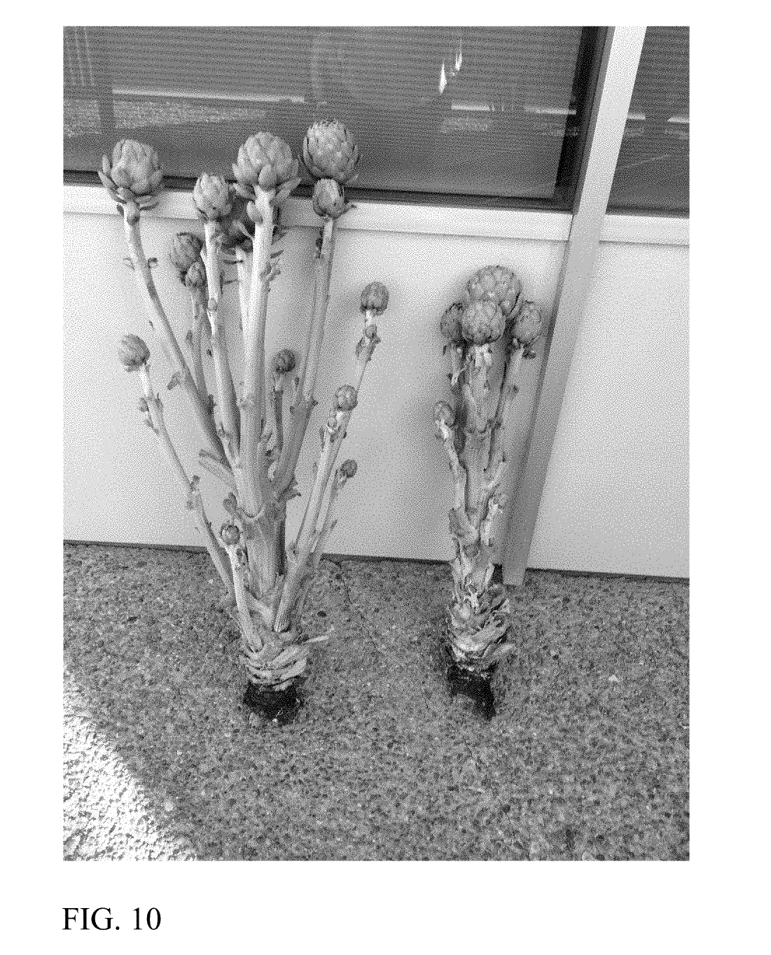

FIG. 11 shows various sized capitula (buds) of ‘PAGA 13-1’.

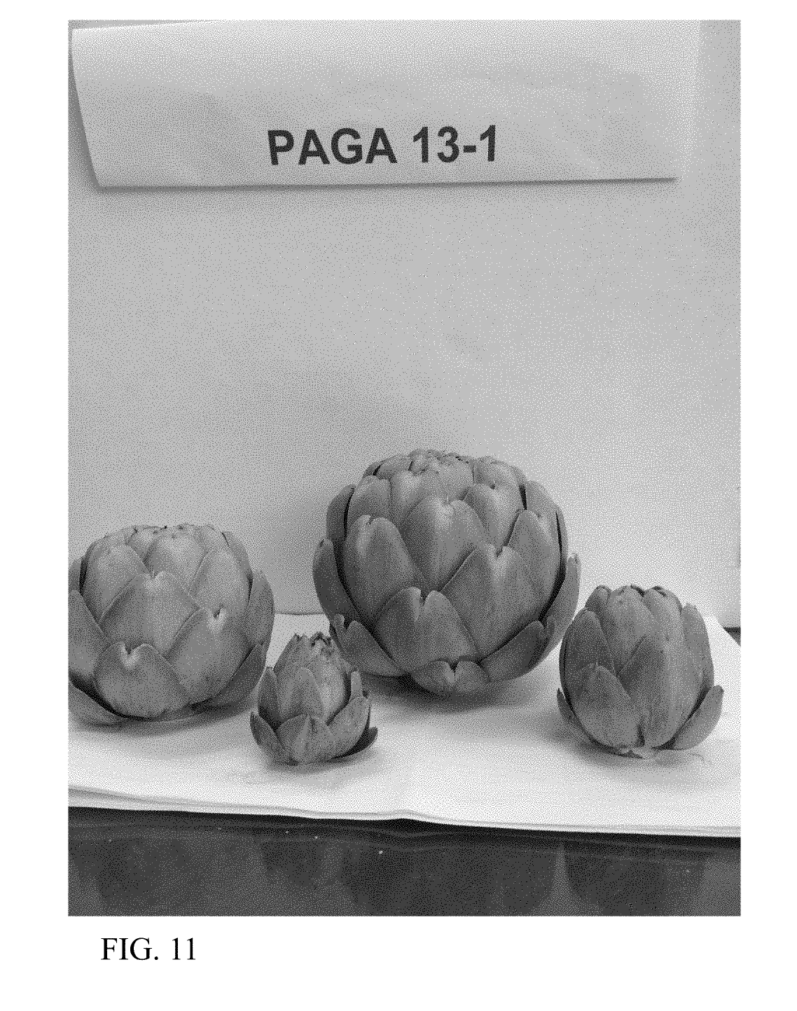

FIG. 12 shows an interior view of a bract of ‘PAGA 13-1’.

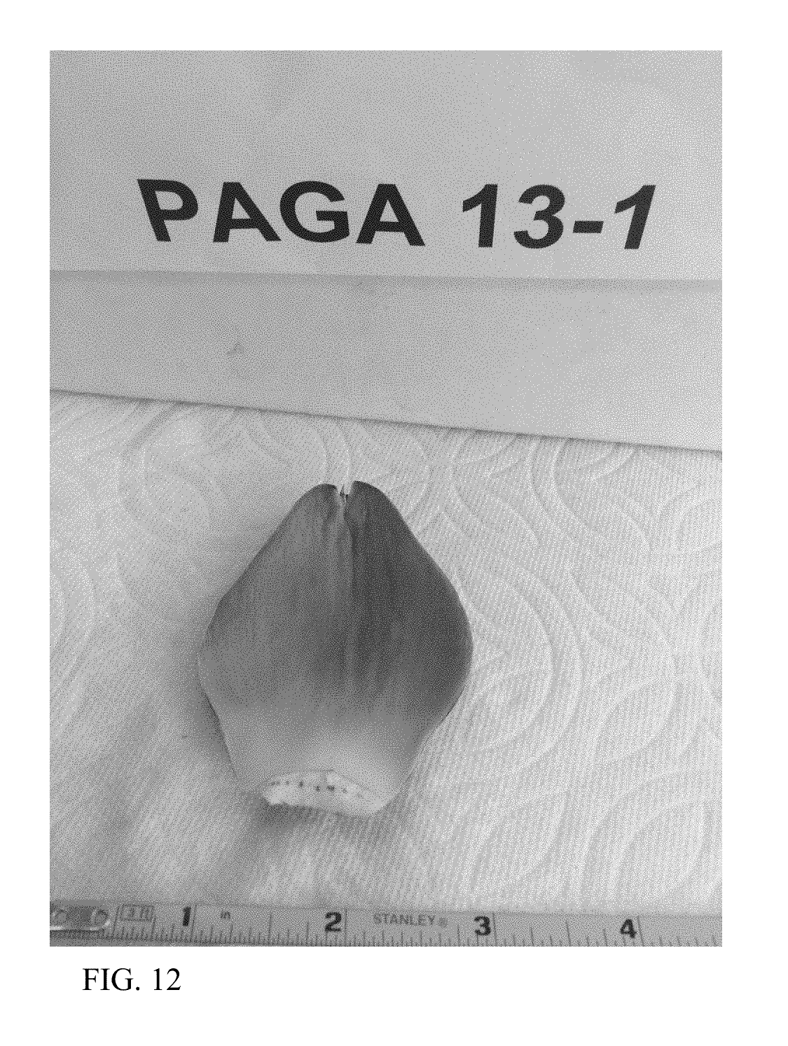

FIG. 13 shows an exterior view of a bract of ‘PAGA 13-1’.

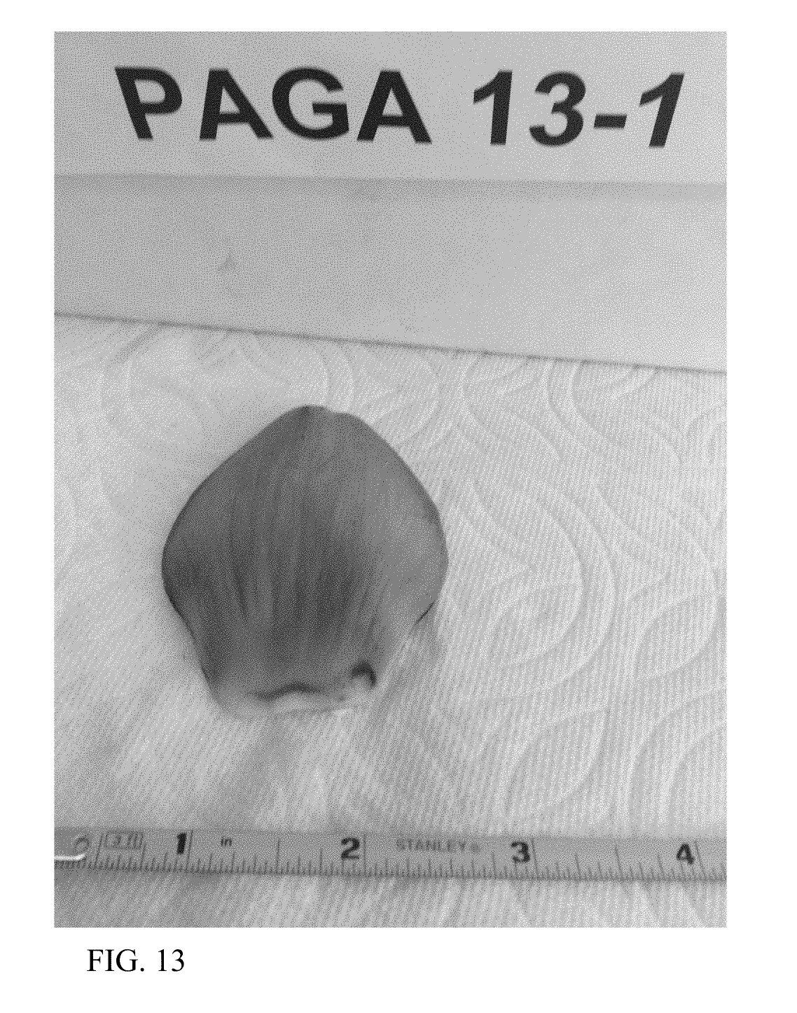

FIG. 14 shows a cross section view of various sized capitula (buds) of “PAGA 13-1’.

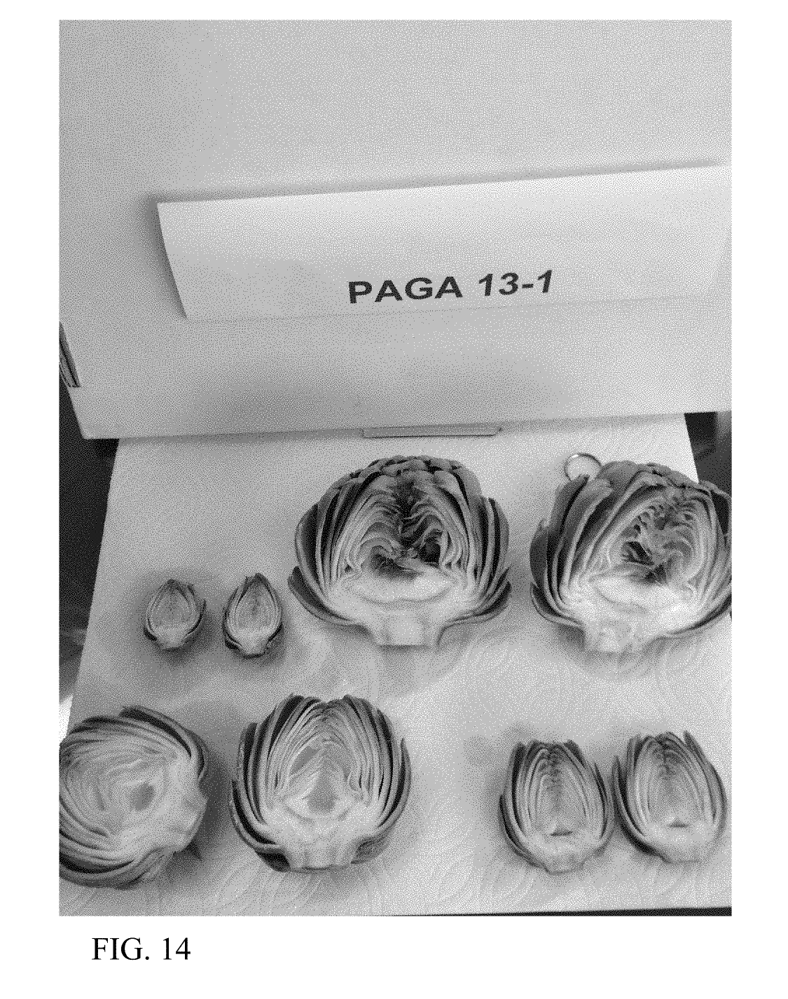

FIG. 15 shows various sized outer and inner bracts of ‘PAGA 13-1’.

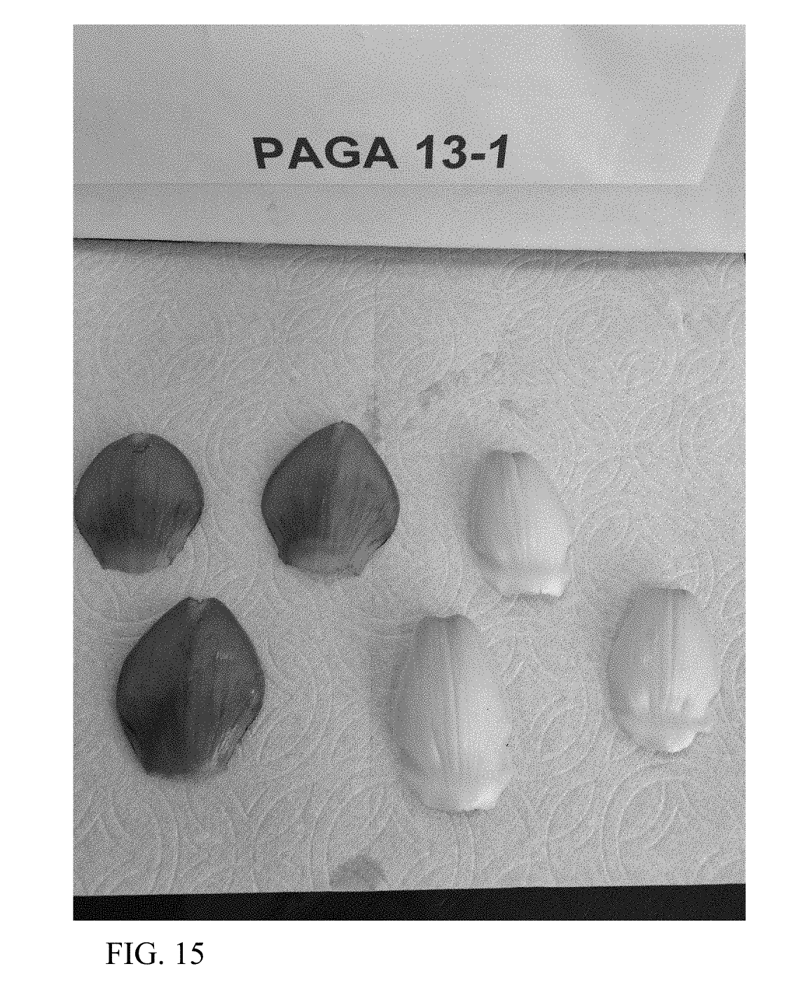

FIG. 16 shows a cauline and a basal rosette leaf of ‘PAGA 13-1’.

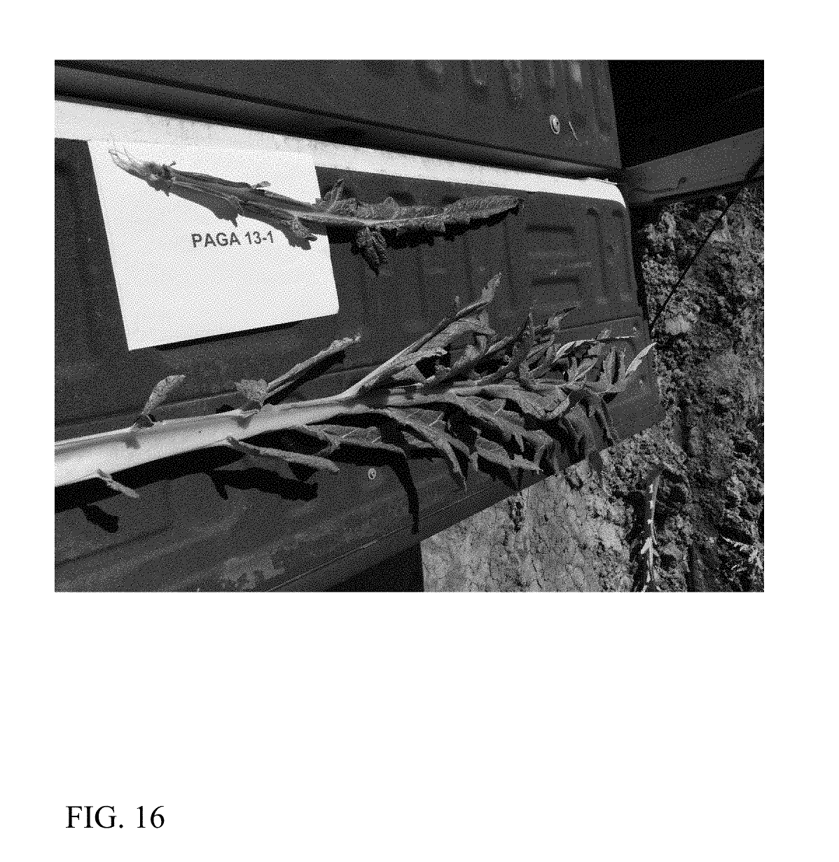

FIG. 17 shows a growing field of ‘PAGA 13-1’ demonstrating its uniformity.

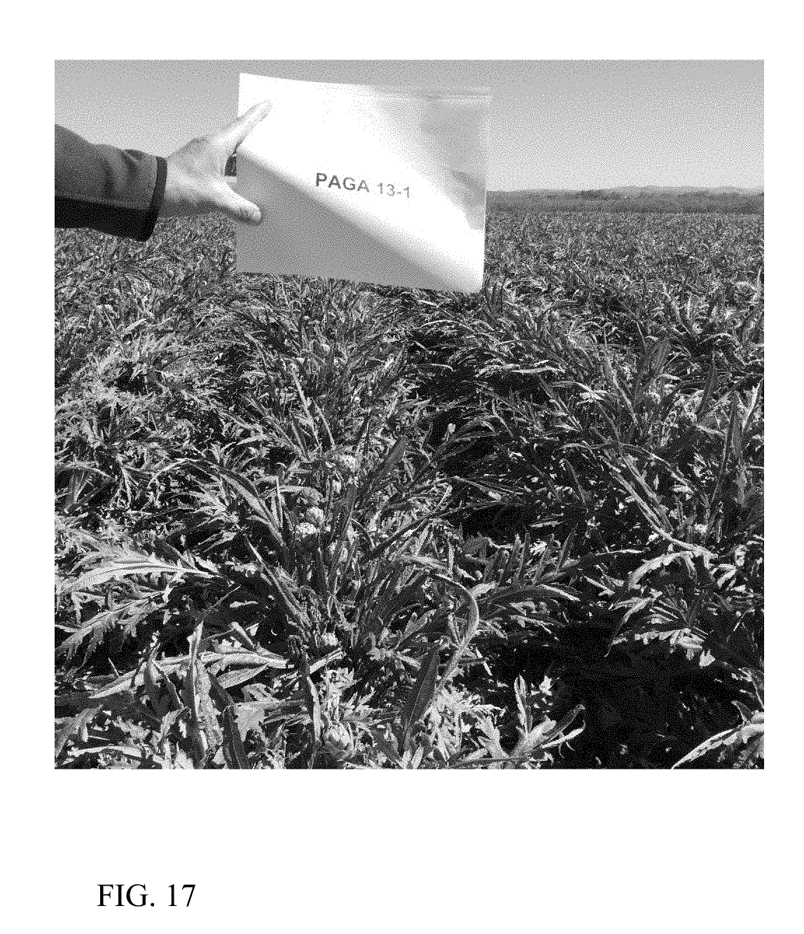

FIG. 18 shows a growing field of parent variety ‘Green Globe’ demonstrating its lack of uniformity.

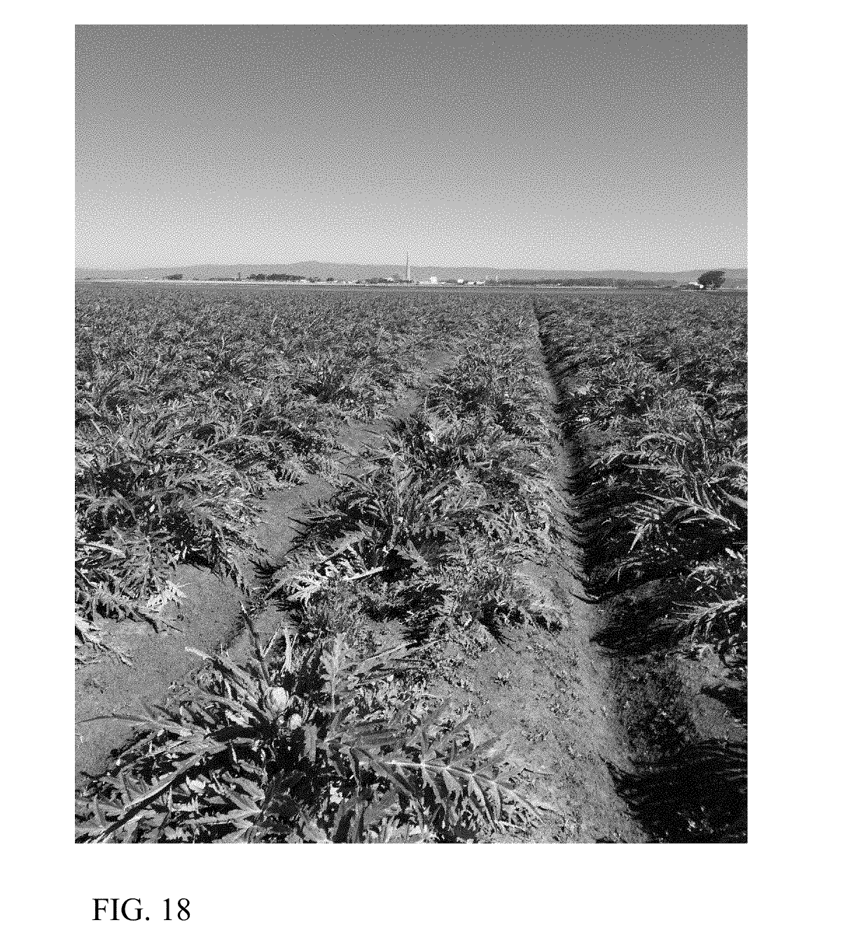

FIG. 19 shows ‘PAGA 13-1’ capitulum (bud) bases showing the differences in purple colored striations on lower bracts.

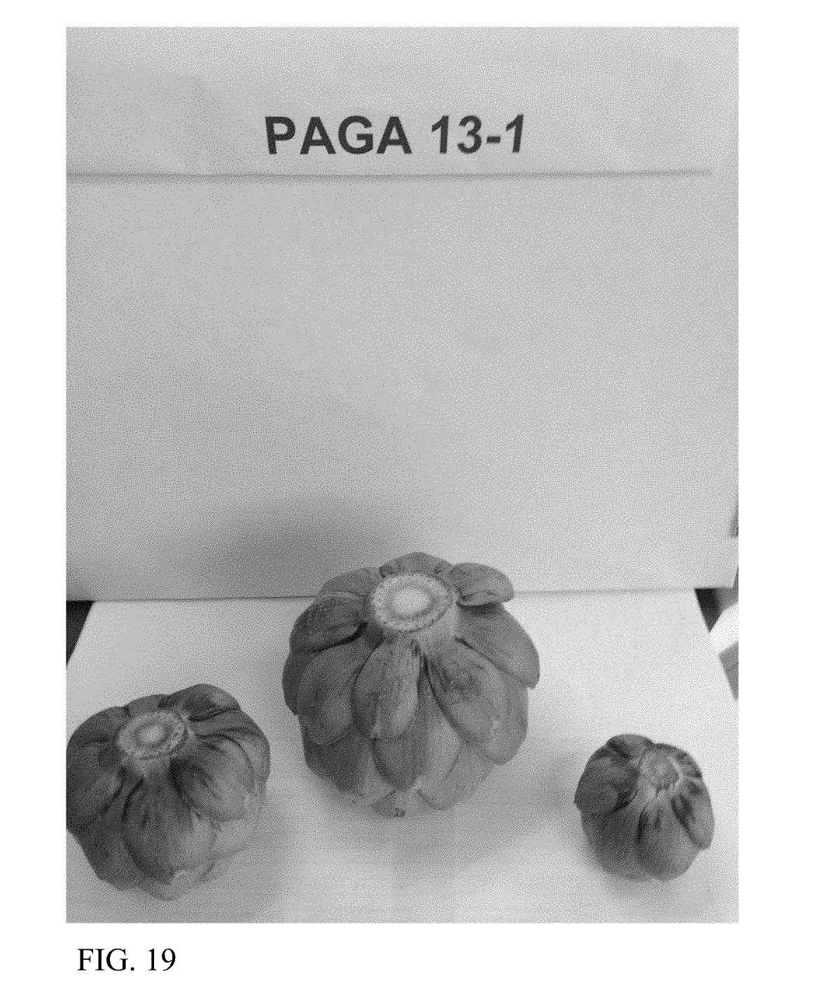

FIG. 20 shows ‘PAGA 13-1’ (right) and ‘Green Globe’ (left) reproductive stems that have been stripped of leaves.

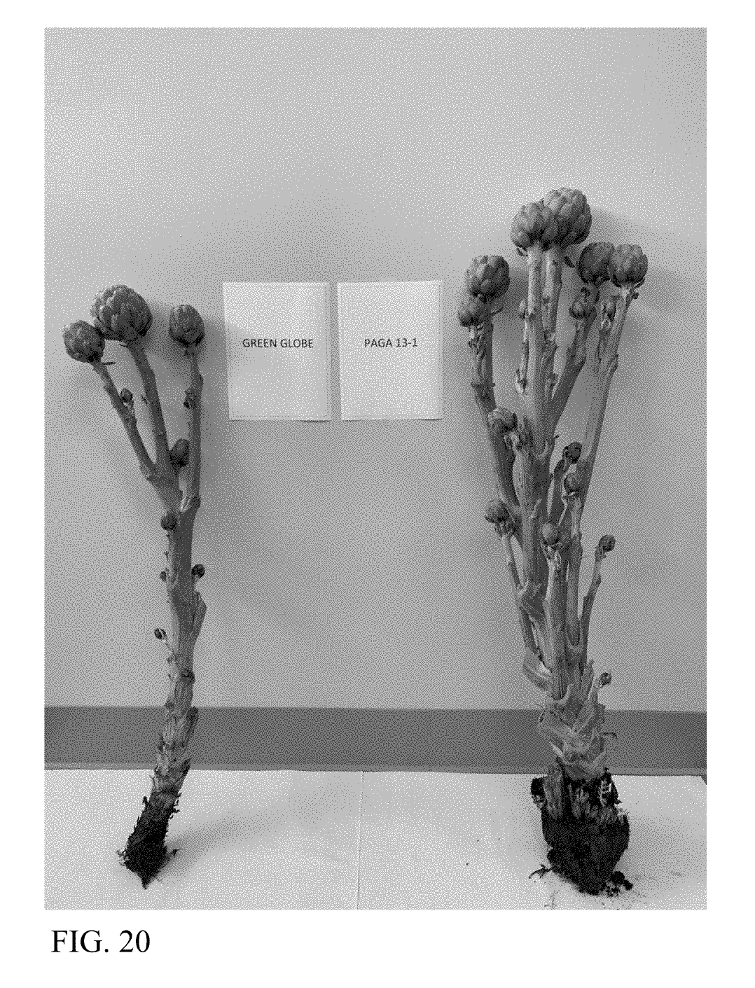

FIG. 21 shows various sized capitula (buds) of ‘PAGA 15-1’.

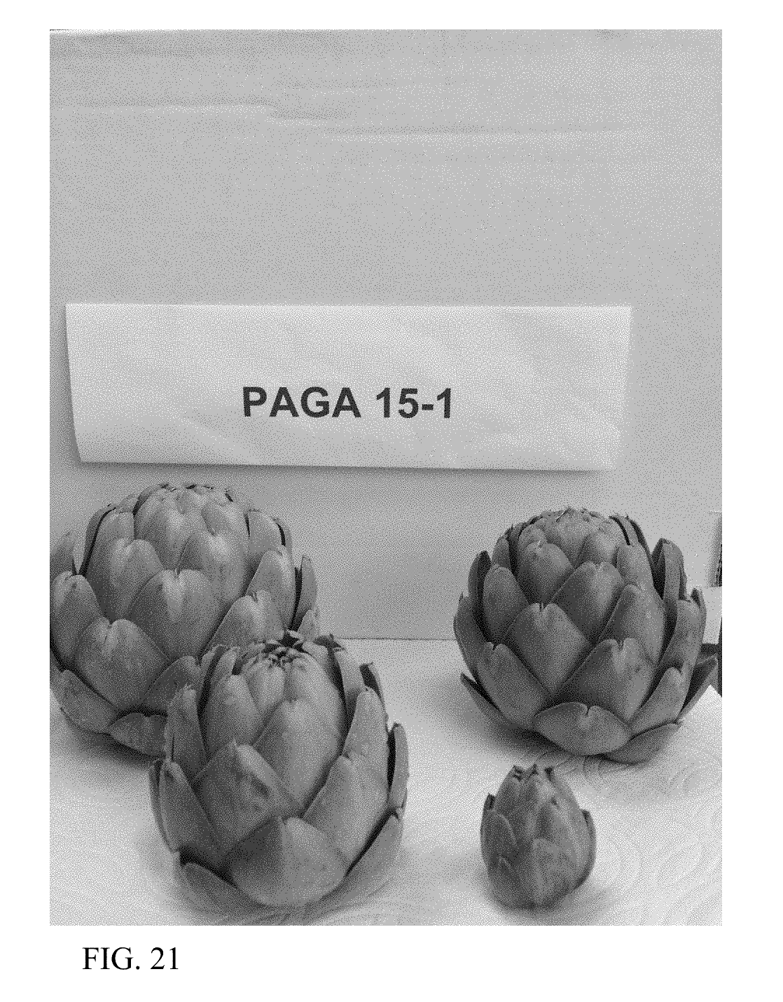

FIG. 22 shows an interior view of a bract of ‘PAGA 15-1’.

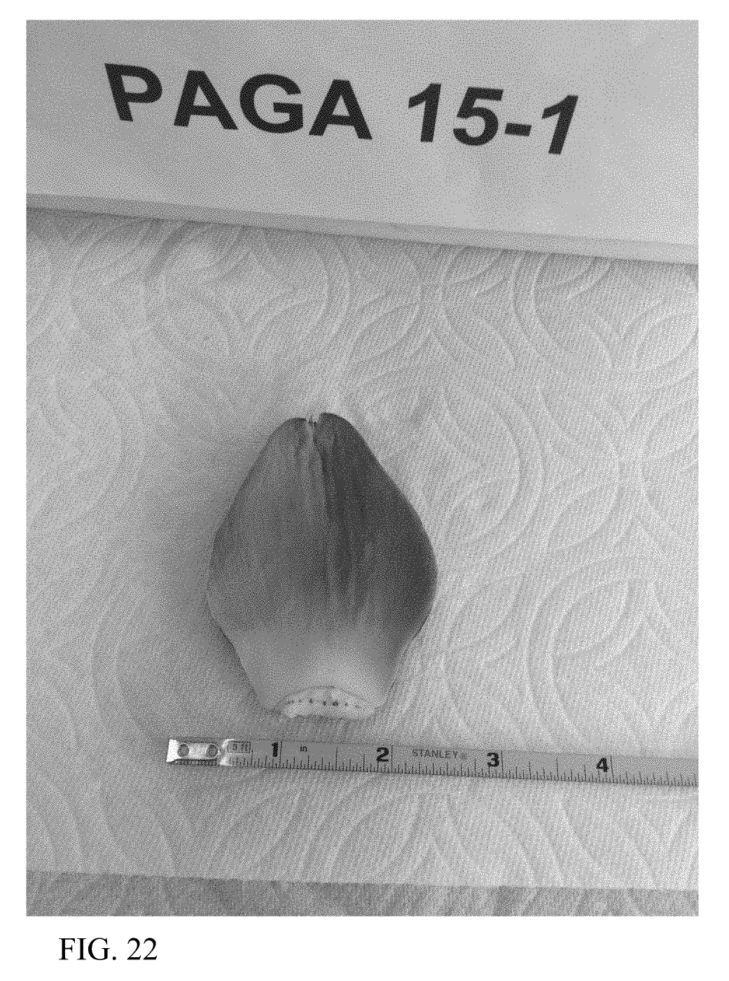

FIG. 23 shows an exterior view of a bract of ‘PAGA 15-1’.

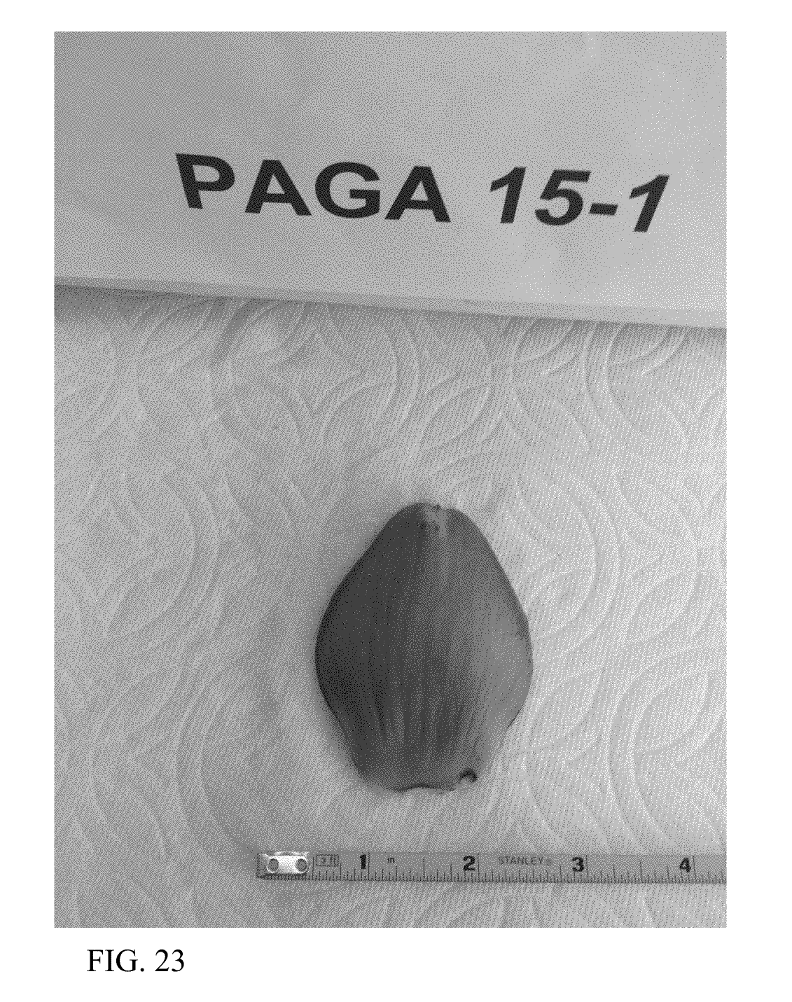

FIG. 24 shows a cross section view of various sized capitula (buds) of “PAGA 15-1’.

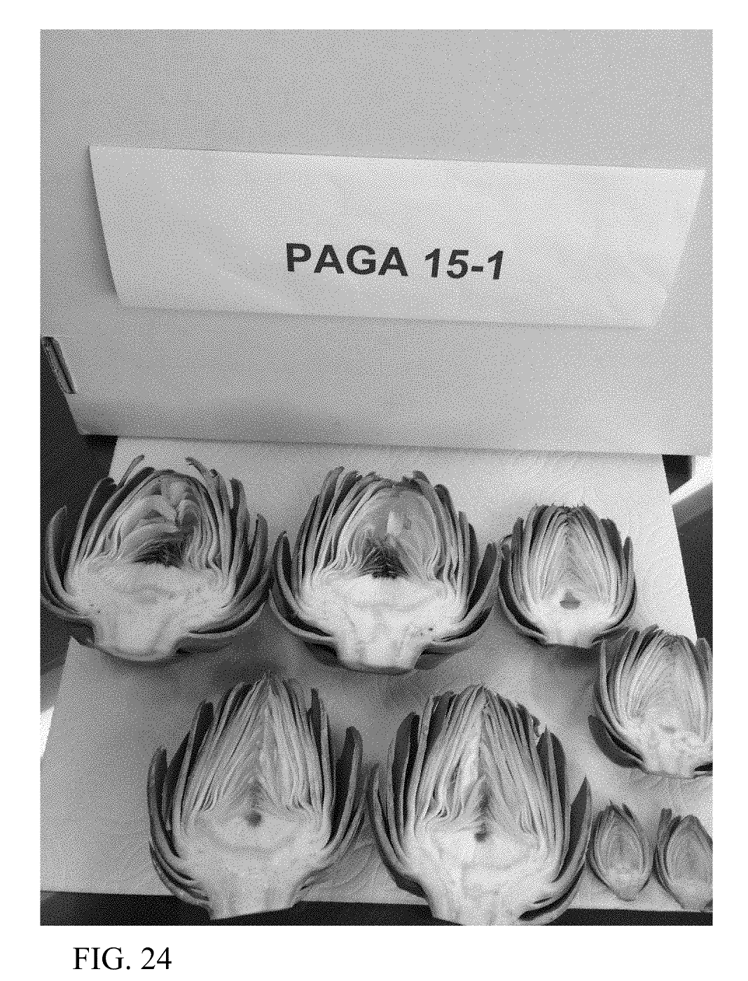

FIG. 25 shows examples of outer and inner bracts of ‘PAGA 15-1’.

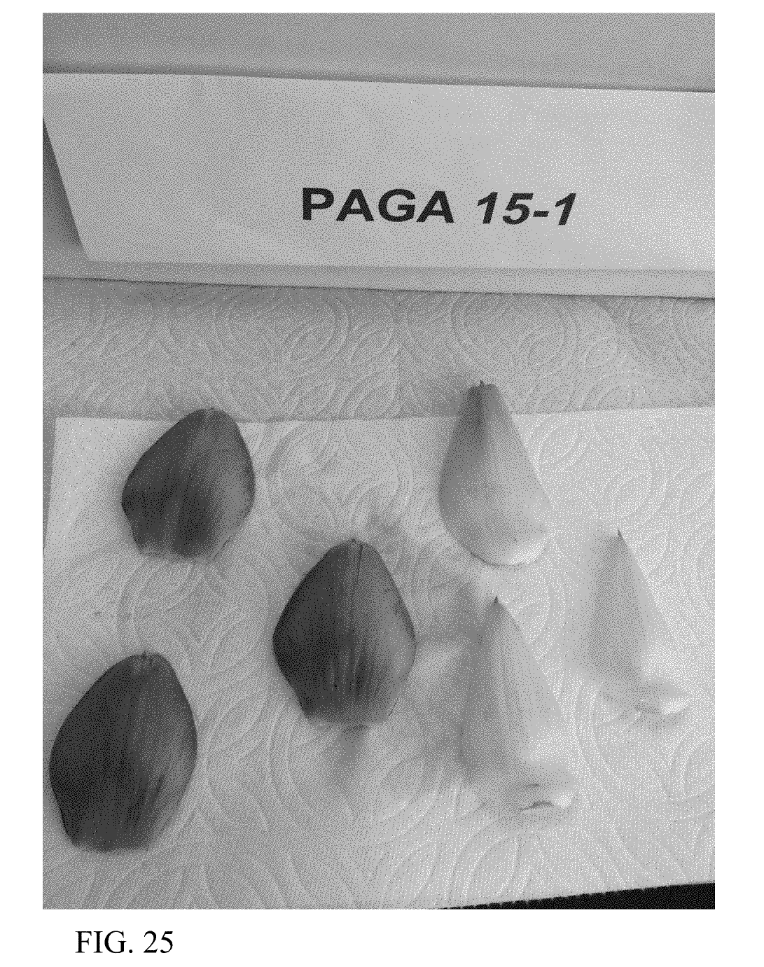

FIG. 26 shows a cauline and a basal rosette leaf of ‘PAGA 15-1’.

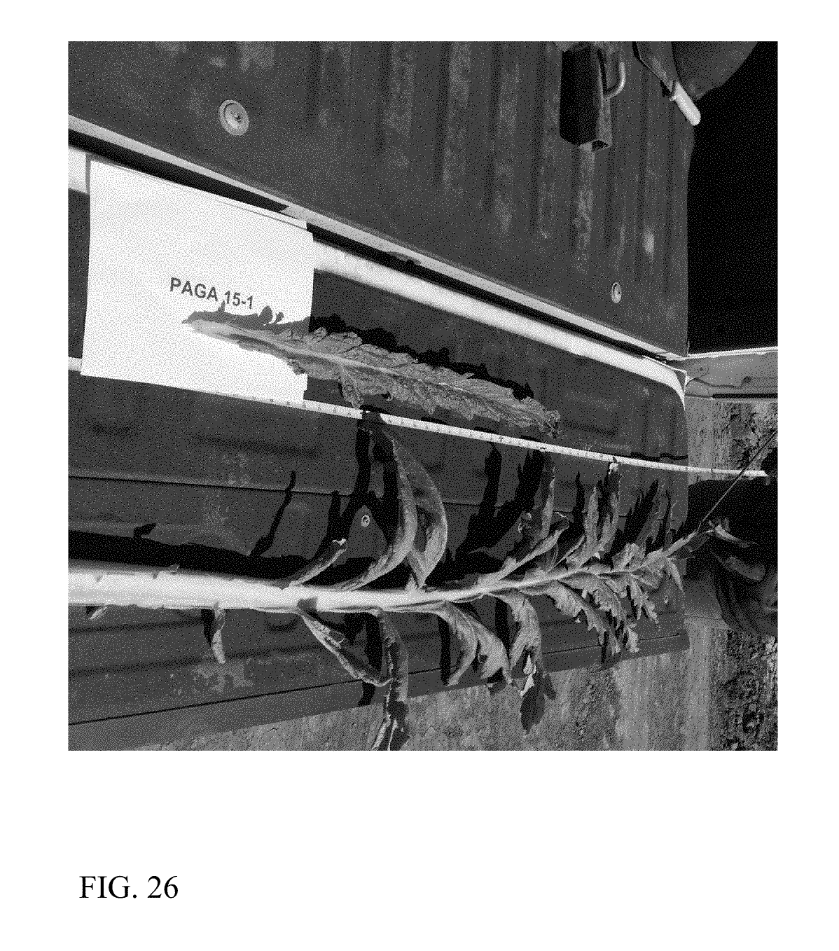

FIG. 27 shows a growing field of ‘PAGA 15-1’ demonstrating its uniformity.

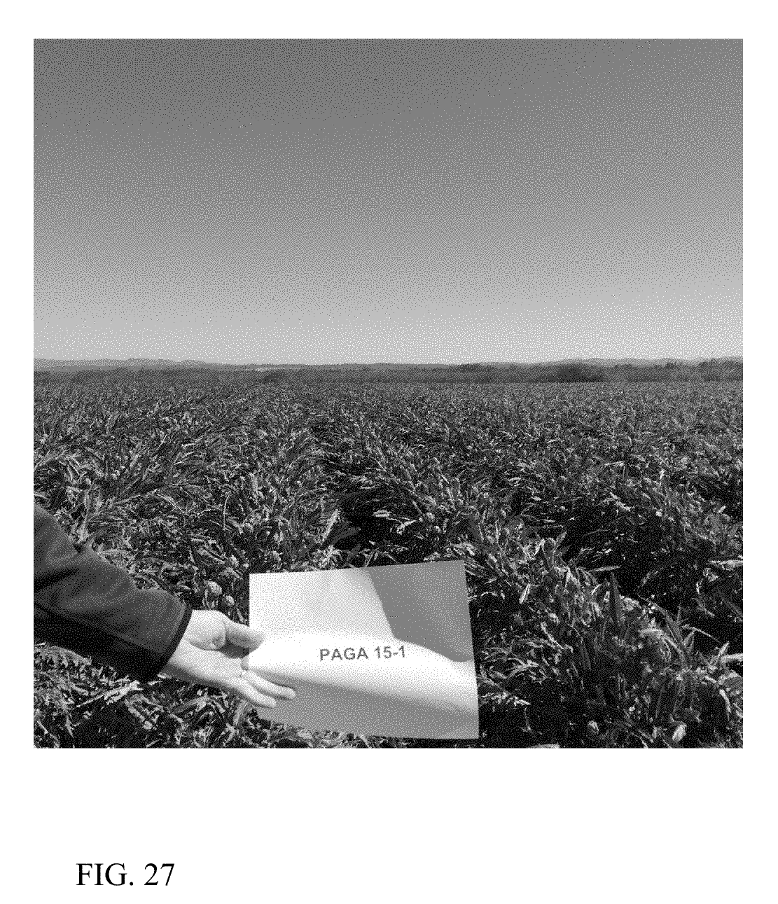

FIG. 28 shows a growing field of the parent variety ‘Green Globe’ demonstrating its lack of uniformity.

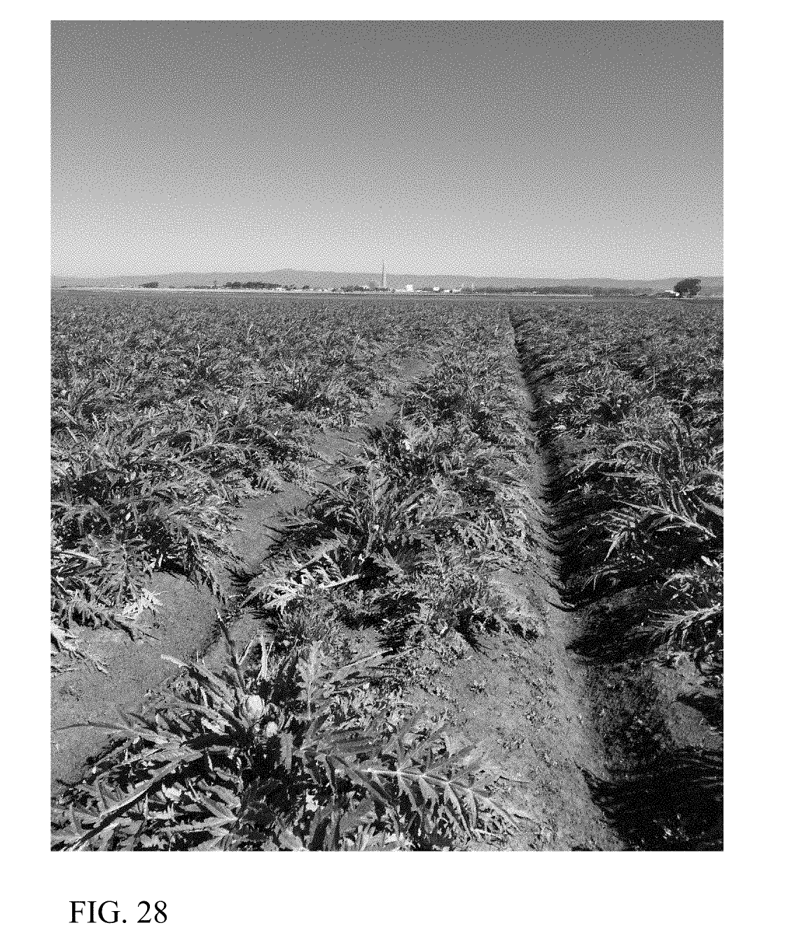

FIG. 29 shows various sized ‘PAGA 15-1’ capitulum (bud) bases showing the differences in purple colored striations on lower bracts.

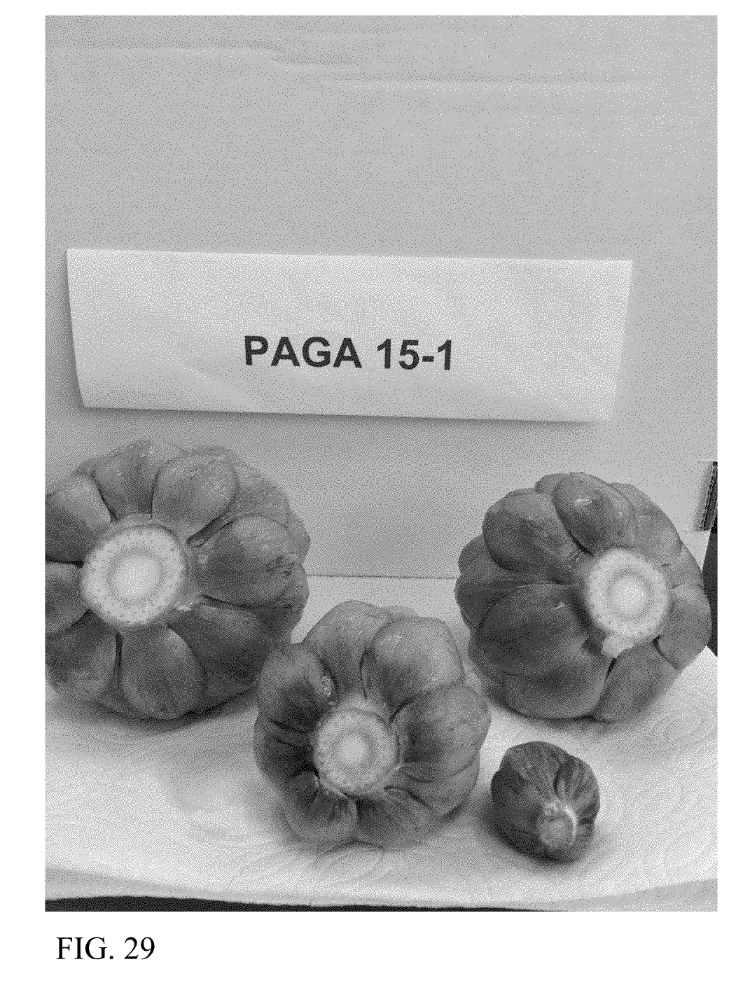

FIG. 30 shows ‘PAGA 15-1’ (right) and ‘Green Globe’ (left) reproductive stems that have been stripped of leaves.

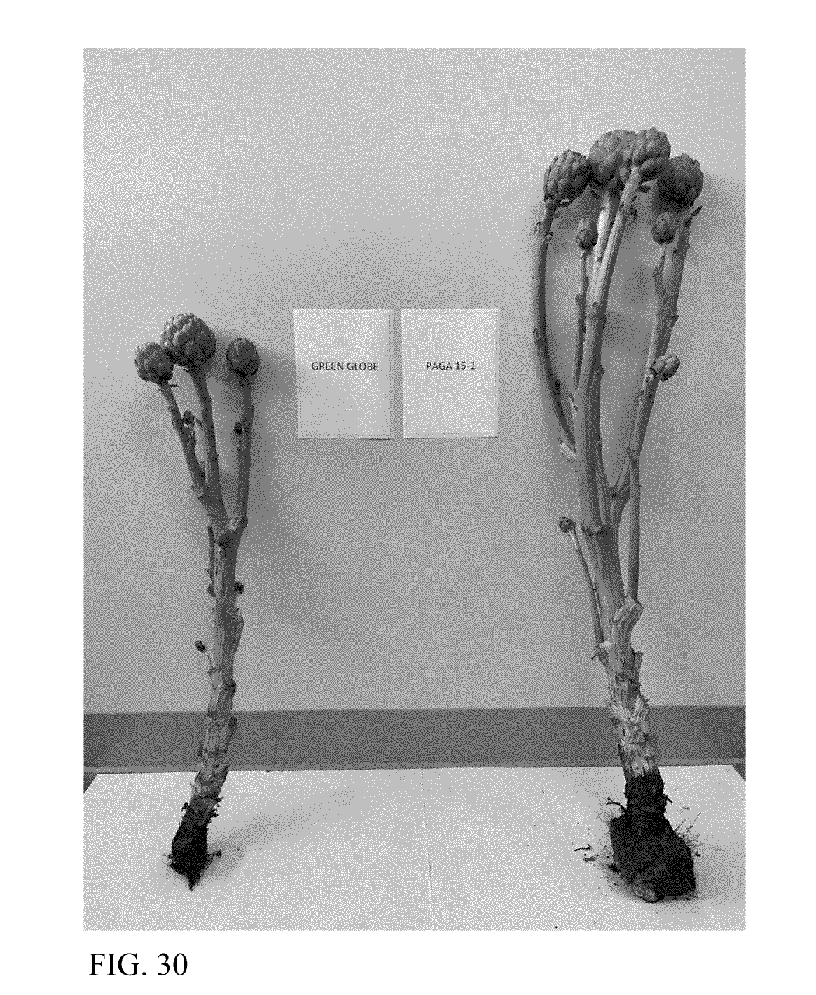

### DETAILED DESCRIPTION OF THE INVENTION

In certain aspects, the present invention is directed to new and improved artichoke plant varieties ‘PAGA G-1’, ‘PAGA 13-1’, and ‘PAGA 15-1’. The artichoke plant varieties described herein are the products of an artichoke plant improvement program conducted over a number of years. The original parent is the unpatented Cynara cardunculus L. var. scolymus variety ‘Green Globe’, commonly known and cultivated in Castroville, Calif. for more than 90 years. This improvement project began with the objective to develop an artichoke plant variety with superior bud production. This included selecting for improvements in both commercial quality of buds, as well as overall bud consistency and large size bud production per plant. Additionally, plants were selected based on their predictable and condensed harvest phase.

The new artichoke plant varieties ‘PAGA G-1’, ‘PAGA 13-1’, and ‘PAGA 15-1’ described herein were discovered after several years of isolating and propagating artichoke plant variety ‘Green Globe’ in Castroville, Calif. From this continual propagation, several selections were identified which exhibited superior yield characteristics as compared to ‘Green Globe’. After identifying the superior quality plants from field conditions, the highest quality plants were selected for induction into laboratory tissue culture reproduction. These tissue-cultured plants were then planted in a test field in Castroville, Calif., where further selections were made among the population. Selections from this field were propagated by tissue culture, with subsequent field selections and tissue culture propagation conducted. The varieties ‘PAGA G-1’, ‘PAGA 13-1’, and ‘PAGA 15-1’ described herein are final products obtained from these selections and tissue culture propagation cycles.

Artichoke plant varieties ‘PAGA G-1’, ‘PAGA 13-1’, and ‘PAGA 15-1’ were subsequently asexually reproduced via tissue culture propagation and regeneration of artichoke plants from plantlets. The present varieties ‘PAGA G-1’, ‘PAGA 13-1’, and ‘PAGA 15-1’ have been found to be stable and reproduce true to type through successive asexual propagations.

### Artichoke Plant Variety ‘PAGA G-1’

In some aspects, the present disclosure relates to a new and distinct artichoke plant variety (Cynara cardunculus L. var. scolymus) which has been denominated as ‘PAGA G-1’. This new artichoke plant variety exhibits increased bud yield, size and plant vigor compared to ‘Green Globe’, and produces a large primary bud, about 7-10 secondary buds, and about 6-14 tertiary buds per primary reproductive shoot. Up to 8 of the primary and secondary buds are 12-15 inches in circumference. Between 4-5 of the secondary and tertiary buds are 10¾-12½ inches in circumference or larger. The remaining buds are generally between 3-10½ inches in circumference. The plants produce between 1 and 5 reproductive shoots, in addition to the primary reproductive shoot, in the first production cycle. These secondary reproductive shoots produce less marketable sized buds than the primary shoot, although cultural practices such as cutting the primary reproductive shoot from the plant after harvest can materially increase the size and number of buds produced by these secondary shoots.

‘PAGA G-1’ exhibits the following distinguishing characteristics when grown under normal horticultural practices in Castroville, Calif.:
1. Large bud size;
2. Primary reproductive shoots produce at least 6 secondary buds, and at least 3 tertiary buds;
3. Plants produce an average of 3 reproductive shoots, in addition to the primary reproductive shoot, during the first production cycle;
4. Bracts and receptacles are wide, and thick;
5. Large diameter peduncle; and
6. Early maturity.

The following detailed descriptions set forth the distinctive characteristics of ‘PAGA G-1’. The data which define these characteristics is based on observations taken in Castroville, Calif. This description is in accordance with UPOV terminology. Color designations, color descriptions, and other phenotypical descriptions may deviate from the stated values and descriptions depending upon variation in environmental, seasonal, climatic, and cultural conditions. ‘PAGA G-1’ has not been observed under all possible environmental conditions. The botanical description of ‘PAGA G-1’ was taken from plants that were 5-6 months old. The indicated values represent averages calculated from measurements of several plants. Color references are primarily to the RHS Colour Chart of The Royal Horticultural Society of London (RHS) (2007 edition). Descriptive terminology follows the Plant Identification Terminology, An Illustrated Glossary, 2^{nd} edition by James G. Harris and Melinda Woolf Harris, unless where otherwise defined.

Classification:
Taxonomic name: Cynara cardunculus L. var. scolymus
Common name: Globe Artichoke
Denomination: ‘PAGA G-1’

Parentage:
Cynara cardunculus var. scolymus L. ‘PAGA G-1’ was discovered through successive plant selections, and tissue culture propagation cycles of Cynara cardunculus L. var. scolymus variety ‘Green Globe’

Plant Characteristics Table:

| | PAGA G-1 | Green Globe |
|---|---|---|
| General information | | |
| Propagation Method: | Tissue culture | Crown division |
| Months of harvest: | Year round with greatest productivity | Year round with greatest |
| | February through June | productivity February through |
| | | June |
| Number of days from | Range: 165-203 Average: 190 (tissue | Range: 161-212 Average 195 |
| planting to first harvest | culture) | (crown division) |
| Duration of harvest cycle: | Average: 75 days | Average: 232 days |
| Plant | | |
| Height range at harvest | 152.4-187.96 cm (60-74 inches) | 119.38-167.64 cm (47-66 |
| (vegetative): | | inches) |
| Height average at harvest | 167.64 cm (66 inches) | 147.32 cm (58 inches) |
| (vegetative): | | |
| Width at harvest (vegetative): | 203.2-243.84 cm (80-96 inches) | 114.3-132.08 cm (45-52 inches) |
| Growth Habit: | Upright, with horizontal spread of | Upright, with horizontal spread |
| | older leaves and shoots | of older leaves and shoots |
| Form: | Arch | Arch |
| Vigor: | Very Good | Moderate |
| Root system: | Thick and fibrous | Thick and fibrous |
| Foliage Density: | Dense, increasing as secondary | Moderately dense |
| | reproductive shoots develop | |
| Primary Reproductive | | |
| Shoot | | |
| Height to primary (terminal) | 152.4 cm-193.04 (60-76 inches) | 106.68-137.16 cm (42-54 |
| bud top: | | inches) |
| Number of leaves | 26-35 (average 31) | 14-28 (average 20) |
| Secondary Reproductive | | |
| Shoots | | |
| Number per plant in first | 1-5 (average 3) | 1-2 (average 1) |
| production cycle: | | |
| Height to primary (terminal) | 40.64-121.92 cm (16-48 inches) | 13.97-76.2 cm (5.5 to 30 inches) |
| bud top: | | |
| Number of leaves | 18-30 (average 23) | 3-16 (average 7.5) |
| Foliage | | |
| Venation: | Reticulate | Reticulate |
| Leaf ratio (L/W): | Approximately 1.1-3.23 | Approximately 1.12-2.28 |
| Upper leaf surface color: | RHS 143A-143B (Green Group) | RHS 141A-141B (Green Group) |
| Lower leaf surface color: | RHS 141D (Green Group) | RHS 136D (Green Group) |
| Leaf texture: | Glabrous and slightly glossy | Glabrous and slightly glossy |
| | appearance in cool, damp conditions | appearance in cool, damp |
| | with interveinal texture. | conditions with interveinal |
| | | texture. |
| Pubescence: | Slightly tomentose on upper surface, | Slightly tomentose on upper |
| | moderately tomentose on lower. More | surface, moderately tomentose |
| | visible in dry/warm conditions. | on lower. More visible in |
| | | dry/warm conditions. |
| Petiole color: | RHS 142B-142C (Green Group) | RHS 142C-142D (Green Group) |
| Petiole and mid rib texture: | Furrowed on upper side with lower | Furrowed on upper side with |
| | side deeply ribbed with ridges | lower side deeply ribbed with |
| | running parallel to the lengthwise | ridges running parallel to the |
| | orientation. Sparsely arachnoid. | lengthwise orientation. Sparsely |
| | | arachnoid. |
| 1. Basal rosette leaves | | |
| Shape: | Deeply pinnatifid with sparsely ciliate | Deeply pinnatifid with sparsely |
| | margins, narrowly trullate overall. | ciliate margins, narrowly trullate |
| | Narrowly acuminate apex is less acute | overall. Narrowly acuminate |
| | than Green Globe. | apex. |
| Size: | Very large, >182.88-78.74 cm (72 × | Very large, >172.72 × 45.72 cm |
| | 31 inches) | (68 × 18 inches) |
| Distance between leaf | 6.35-11.43 cm (2½-4½ inches) | 6.35-11.43 cm (2½-4½ |
| division: | | inches) |
| Division indentation length: | 16.51-41.275 cm (6½-16¼ | 16.51-41.275 cm (6½-16¼ |
| | inches) | inches) |
| Petiole length: | 30.48-57.15 cm (12-22½ inches) | 30.48-48.26 cm (12-19 inches) |
| Petiole width: | 3.175-9.525 cm (1¼-3¾ inches) | 3.175-6.985 cm (1¼-2¾ |
| | | inches) |
| Petiole thickness: | 0.635-1.905 cm (¼-¾ inches) | 0.635-1.905 cm (¼-¾ inches) |
| 2. Cauline leaves | | |
| Shape: | Ensiform overall, uneven and sparse | Ensiform overall, uneven and |
| | serrations with narrowly acuminate | sparse serrations with narrowly |
| | apex. Somewhat undulate and dente | acuminate apex. Somewhat |
| | and sparsely ciliate at margins. | undulate and dente and sparsely |
| | | ciliate at margins. |
| Number of leaves: | Approximately 85 | Approximately 73 |
| Size: | 17.78-60.96 cm (7-24 inches) | 17.78-60.96 cm (7-24 inches) |
| Stem (primary reproductive | | |
| stem system) | | |
| Length (ground level to base | 152.4 cm-193.04 (60-76 inches) | 106.68-127.0 cm (42-50 inches) |
| of terminal capitulum (bud)): | | |
| Circumference: (base of | 7.62-13.97 cm (3-5½ inches) | 7.62-12.065 cm (3-4.75 inches) |
| terminal capitulum (bud): | | |
| Circumference (base of | Average: 27.94 cm (11 inches) | Average: 24.13 cm (9½ |
| stem): | | inches) |
| Exterior texture: | Vertically ribbed, pubescent | Vertically ribbed, pubescent |
| Stem habit: | Upright, caulescent | Upright, caulescent |
| Pubescence: | Sparsely arachnoid | Sparsely arachnoid |
| Color: | RHS 142A-142B | RHS 142B-142C |
| Involucre bracts | | |
| Arrangement: | Imbricate | Imbricate |
| Quantity per Capitulum | 52 outer-35 inner | 52 outer-35 inner |
| (average): | | |
| Outer bract | | |
| Shape: | Predominately ovate with flattened | Predominately ovate with |
| | base | flattened base |
| Apex: | Retuse | Retuse |
| Margin: | Entire | Entire |
| Base: | Slightly rounded | Slightly rounded |
| Texture: | Firm, thick and fleshy | Firm, thick and fleshy |
| Color: | RHS 142A-142B (Green Group) | RHS 141C-141D (Green Group) |
| Surface (outer/inner): | Glabrous | Glabrous |
| Spinosity: | Moderate frequency, woody | Moderate frequency, woody |
| Length of spines: | 1-5 mm (0.04-0.2 inches) | 1-5 mm (0.04-0.2 inches) |
| Striation at base of lower | Very minor - only present on the | Present on approximately 20% |
| bracts: | margins of a few lower outside bracts. | of lower outside bracts. |
| | More apparent on immature buds. | |
| Striation color: | RHS 77A (Purple-Violet Group) | RHS 77A (Purple-Violet Group) |
| Inner bract | | |
| Shape: | Lanceolate with caudate tip | Lanceolate with caudate tip |
| Apex: | Retuse | Retuse |
| Margin: | Entire | Entire |
| Texture: | Firm, thinner than outer bract, fleshy | Firm, thinner than outer bract, |
| | | fleshy |
| Base: | Slightly rounded | Slightly rounded |
| Color: | RHS 150B-150C (Green Group) | RHS 150B-150C (Green Group) |
| Surface (outer/inner): | Glabrous | Glabrous |
| Spinosity: | Finer than outer bract, more frequent | Finer than outer bract, more |
| | | frequent |
| Length of spines: | 1-2 mm (0.04-0.079 inches) | 1-2 mm (0.04-0.079 inches) |
| Capitulum (head/bud) - | | |
| when ready for harvest as | | |
| edible bud | | |
| Color: | RHS 142A-142B (Green Group) | RHS 141C-141D (Green Group) |
| Shape: | Globose to ovoid | Globose to ovoid |
| Apex: | Broadly rounded to obtuse | Broadly rounded to obtuse |
| Base: | Truncate to rounded | Truncate to rounded |
| Form: | Tightly compact | Tightly compact |
| Texture: | Firm | Firm |
| Receptacle shape: | Concavo-convex | Concavo-convex |
| 1. Primary (Terminal) | | |
| Capitulum | | |
| Number per primary shoot | 1 | 1 |
| Circumference: | 34.925-43.18 cm (13¾-17 inches) | 32.385-38.1 cm (12¾-15 |
| | | inches) |
| Weight: | Average 470 grams (1.03 lbs) | Average 425 grams (.94 lbs.) |
| Height: | Average 10.795 cm (4¼ inches) | Average 10.16 cm (4 inches) |
| Diameter: | 12.7-15.24 cm (5-6 inches) | 11.43-12.7 cm (4½ -5 inches) |
| 2. Secondary capitulum | | |
| Number per primary shoot | 7-10 | 3-5 |
| Circumference | 7.62-38.1 cm (3-15 inches) | 7.62-38.1 cm (3-15 inches) |
| Weight: | Average 321 grams (.707 lbs) | Average 237 grams (.52 lbs.) |
| Height: | Average 9.525 cm (3¾ inches) | Average 8.255 cm (3¼ inches) |
| Diameter: | 2.54-12.7 cm (1-5 inches) | 2.54-12.7 cm (1-5 inches) |
| 3. Tertiary capitulum | | |
| Number per primary shoot | 6-14 | 2-5 |
| Circumference | 7.62-30.48 cm (3-12 inches) | 7.62-27.31 cm (3-10¾ inches) |
| Weight: | Average 68 grams (0.14991 lbs) | Average 44 grams (0.0970 lbs) |
| Height: | Average 8.46 cm (3⅓ inches) | Average 6.78 cm (2⅔ inches) |
| Diameter: | 5.08-11.43 cm (2-4½ inches) | 5.08-10.795 cm (2-4¼ inches) |
| Receptacle (heart) - when | | |
| ready for harvest and edible | | |
| Shape: | Concavo-convex | Concavo-convex |
| Circumference: | 8.89-25.4 cm (3½-10 inches) | 8.89-25.4 cm (3½-10 inches) |
| Color: | RHS 150D (Green group) | RHS 150D (Green group) |
| Texture: | Firm | Firm |
| Size: | Very thick and wide when compared | Very thick and wide when |
| | to other globe varieties | compared to other globe |
| | | varieties |
| Peduncle | | |
| Shape: | Columnar | Columnar |
| Length: | 2.54-101.6 cm (1-40 inches) | 2.54-76.2 cm (1-30 inches) |
| Diameter: | .635-4.445 cm (¼-1¾ inches) | .635-3.81 cm (¼-1½ inches) |
| Color: | RHS 142A-142B | RHS 142B-142C |
| Strength: | Strong and rigid | Strong and rigid |
| Surface: | Glabrous, slightly pubescent with | Glabrous, slightly pubescent |
| | shallow vertical ribbing | with shallow vertical ribbing |

### Artichoke Plant Variety ‘PAGA 13-1’

In some aspects, the present disclosure relates to a new and distinct artichoke plant variety (Cynara cardunculus L. var. scolymus) which has been denominated as ‘PAGA 13-1’. This new artichoke plant variety exhibits increased bud yield, size and plant vigor compared to ‘Green Globe’, and produces a large primary bud, about 10-19 secondary buds, and about 10-16 tertiary buds per primary reproductive shoot. Up to 6 of the primary and secondary buds are 12-15 inches in circumference. Between 4-5 of the secondary and tertiary buds are 10¾-12½ inches in circumference or larger. The remaining buds are generally between 3-10½ inches in circumference. The plants produce between 4 and 10 reproductive shoots, in addition to the primary reproductive shoot, in the first production cycle. These secondary reproductive shoots produce less marketable sized buds than the primary shoot, although cultural practices such as cutting the primary reproductive shoot from the plant after harvest can materially increase the size and number of buds produced by these secondary shoots.

‘PAGA 13-1’ exhibits the following distinguishing characteristics when grown under normal horticultural practices in Castroville, Calif.:
1. Large bud size;
2. Primary reproductive shoots produce at least 10 secondary buds, and at least 10 tertiary buds;
3. Plants produce an average of 6 reproductive shoots, in addition to the primary reproductive shoot, during the first production cycle;
4. Bracts and receptacles are wide, and thick;
5. Large diameter peduncle; and
6. Late maturity.

The following detailed descriptions set forth the distinctive characteristics of ‘PAGA 13-1’. The data which define these characteristics is based on observations taken in Castroville, Calif. This description is in accordance with UPOV terminology. Color designations, color descriptions, and other phenotypical descriptions may deviate from the stated values and descriptions depending upon variation in environmental, seasonal, climatic, and cultural conditions. ‘PAGA 13-1’ has not been observed under all possible environmental conditions. The botanical description of ‘PAGA 13-1’ was taken from plants that were 5-6 months old. The indicated values represent averages calculated from measurements of several plants. Color references are primarily to the RHS Colour Chart of The Royal Horticultural Society of London (RHS) (2007 edition). Descriptive terminology follows the Plant Identification Terminology, An Illustrated Glossary, 2^{rd} edition by James G. Harris and Melinda Woolf Harris, unless where otherwise defined.

Classification:
Taxonomic name: Cynara cardunculus L. var. scolymus
Common name: Globe Artichoke
Denomination: ‘PAGA 13-1’

Parentage:
Cynara cardunculus var. scolymus L. ‘PAGA 13-1’ was discovered through successive plant selections, and tissue culture propagation cycles of Cynara cardunculus L. var. scolymus variety ‘Green Globe’.

Plant Characteristics Table:

| | PAGA 13-1 | Green Globe |
|---|---|---|
| General information | | |
| Propagation Method: | Tissue culture | Crown division |
| Months of harvest: | Year round with greatest | Year round with greatest |
| | productivity April through July | productivity February through |
| | | June |
| Number of days from | Range: 165-203 Average: 190 | Range: 161-212 Average 195 |
| planting to first harvest | (tissue culture) | (crown division) |
| Duration of harvest cycle: | Average: 69 days | Average: 232 days |
| Plant | | |
| Height range at harvest | 162.56-198.12 cm (64-78 inches) | 119.38-167.64 cm (47-66 inches) |
| (vegetative): | | |
| Height average at harvest | 185.42 cm (73 inches) | 147.32 cm (58 inches) |
| (vegetative): | | |
| Width at harvest (vegetative): | 152.4-284.48 cm (60-112 inches) | 114.3-132.08 cm (45-52 inches) |
| Growth Habit: | Upright, with horizontal spread of | Upright, with horizontal spread of |
| | older leaves and shoots | older leaves and shoots |
| Form: | Arch | Arch |
| Vigor: | Very Good | Moderate |
| Root system: | Thick and fibrous | Thick and fibrous |
| Foliage Density: | Dense, increasing as secondary | Moderately dense |
| | reproductive shoots develop | |
| Primary Reproductive | | |
| Shoot | | |
| Height to primary (terminal) | 162.56-198.12 cm (64-78 inches) | 106.68-137.16 cm (42-54 inches) |
| bud top: | | |
| Number of leaves (rosette) | 25-33 (average 31) | 14-28 (average 20) |
| Secondary Reproductive | | |
| Shoots | | |
| Number per plant in first | 4-10 (average 6) | 1-2 (average 1) |
| production cycle: | | |
| Height to primary (terminal) | 40.64-121.92 cm (16-48 inches) | 13.97-76.2 cm (5.5 to 30 inches) |
| bud top: | | |
| Number of leaves | 5-27 (average 7) | 3-16 (average 7.5) |
| Foliage | | |
| Venation: | Reticulate | Reticulate |
| Leaf ratio (L/W): | Approximately 1.1-3.23 | Approximately 1.12-2.28 |
| Upper leaf surface color: | RHS 143A-143B (Green Group) | RHS 141A-141B (Green Group) |
| Lower leaf surface color: | RHS 141D (Green Group) | RHS 136D (Green Group) |
| Leaf texture: | Glabrous and slightly glossy | Glabrous and slightly glossy |
| | appearance in cool, damp | appearance in cool, damp |
| | conditions with interveinal texture. | conditions with interveinal texture. |
| Pubescence: | Slightly tomentose on upper | Slightly tomentose on upper |
| | surface, moderately tomentose on | surface, moderately tomentose on |
| | lower. More visible in dry/warm | lower. More visible in dry/warm |
| | conditions. | conditions. |
| Petiole color: | RHS 142B-142C (Green Group) | RHS 142C-142D (Green Group) |
| Petiole and mid rib texture: | Furrowed on upper side with lower | Furrowed on upper side with lower |
| | side deeply ribbed with ridges | side deeply ribbed with ridges |
| | running parallel to the lengthwise | running parallel to the lengthwise |
| | orientation. Sparsely arachnoid. | orientation. Sparsely arachnoid. |
| 1. Basal rosette leaves | | |
| Shape: | Deeply pinnatifid with sparsely | Deeply pinnatifid with sparsely |
| | ciliate margins, narrowly trullate | ciliate margins, narrowly trullate |
| | overall. Narrowly acuminate apex | overall. Narrowly acuminate apex. |
| | is less acute than Green Globe. | |
| Size: | Very large, >198.12-78.74 cm (78 × | Very large, >172.72 × 45.72 cm |
| | 31 inches) | (68 × 18 inches) |
| Distance between leaf | 6.35-12.7 cm (2½-5 inches) | 6.35-11.43 cm (2½-4½ |
| division: | | inches) |
| Division indentation length: | 16.51-41.275 cm (6½-16¼ | 16.51-41.275 cm (6½-16¼ |
| | inches) | inches) |
| Petiole length: | 30.48-57.15 cm (12-22½ inches) | 30.48-48.26 cm (12-19 inches) |
| Petiole width: | 3.175-9.525 cm (1¼-3¾ | 3.175-6.985 cm (1¼-2¾ |
| | inches) | inches) |
| Petiole thickness: | 0.635-1.905 cm (¼-¾ inches) | 0.635-1.905 cm (¼-¾ inches) |
| 2. Cauline leaves | | |
| Shape: | Ensiform overall, uneven and | Ensiform overall, uneven and |
| | sparse serrations with narrowly | sparse serrations with narrowly |
| | acuminate apex. Somewhat | acuminate apex. Somewhat |
| | undulate and dente and sparsely | undulate and dente and sparsely |
| | ciliate at margins. | ciliate at margins. |
| Number of leaves: | Approximately 93 | Approximately 73 |
| Size: | 17.78-60.96 cm (7-24 inches) | 17.78-60.96 cm (7-24 inches) |
| Stem (primary reproductive | | |
| stem system) | | |
| Length (ground level to base | 162.56-198.12 cm (64-78 inches) | 106.68-127.0 cm (42-50 inches) |
| of terminal capitulum (bud)): | | |
| Circumference: (base of | 7.62-13.97 cm (3-5½ inches) | 7.62-12.065 cm (3-4.75 inches) |
| terminal capitulum (bud): | | |
| Circumference (base of | Average: 36.83 cm (14½ inches) | Average: 24.13 cm (9½ inches) |
| stem): | | |
| Exterior texture: | Vertically ribbed, pubescent | Vertically ribbed, pubescent |
| Stem habit: | Upright, caulescent | Upright, caulescent |
| Pubescence: | Sparsely arachnoid | Sparsely arachnoid |
| Color: | RHS 142A-142B | RHS 142B-142C |
| Involucre bracts | | |
| Arrangement: | Imbricate | Imbricate |
| Quantity per Capitulum | 62 outer-37 inner | 52 outer-35 inner |
| (average): | | |
| Outer bract | | |
| Shape: | Predominately ovate with flattened | Predominately ovate with flattened |
| | base | base |
| Apex: | Retuse with the tips curving down | Retuse |
| | inside the capitulum | |
| Margin: | Entire | Entire |
| Base: | Slightly rounded | Slightly rounded |
| Texture: | Firm, thick and fleshy | Firm, thick and fleshy |
| Color: | RHS 142A-142B (Green Group) | RHS 141C-141D (Green Group) |
| Surface (outer/inner): | Glabrous | Glabrous |
| Spinosity: | Moderate frequency, woody | Moderate frequency, woody |
| Length of spines: | 1-5 mm (0.04-0.2 inches) | 1-5 mm (0.04-0.2 inches) |
| Striation at base of lower | Minor - present on lower outside | Present on approximately 20% of |
| bracts: | bracts | lower outside bracts |
| Striation color: | RHS 77A (Purple-Violet Group) | RHS 77A (Purple-Violet Group) |
| Inner bract | | |
| Shape: | Lanceolate with caudate tip | Lanceolate with caudate tip |
| Apex: | Retuse with the tips curving down | Retuse |
| | inside the capitulum | |
| Margin: | Entire | Entire |
| Texture: | Firm, thinner than outer bract, | Firm, thinner than outer bract, |
| | fleshy | fleshy |
| Base: | Slightly rounded | Slightly rounded |
| Color: | RHS 150B-150C (Green Group) | RHS 150B-150C (Green Group) |
| Surface (outer/inner): | Glabrous | Glabrous |
| Spinosity: | Finer than outer bract, more | Finer than outer bract, more |
| | frequent | frequent |
| Length of spines: | 1-2 mm (0.04-0.079 inches) | 1-2 mm (0.04-0.079 inches) |
| Capitulum (head/bud) - | | |
| when ready for harvest as | | |
| edible bud | | |
| Color: | RHS 142A-142B (Green Group) | RHS 141C-141D (Green Group) |
| Shape: | Sub-globose with slightly flattened | Globose to ovoid |
| | base and top | |
| Apex: | Flat to concave | Broadly rounded to obtuse |
| Base: | Truncate to rounded | Truncate to rounded |
| Form: | Tightly compact | Tightly compact |
| Texture: | Firm | Firm |
| Receptacle shape: | Concavo-convex | Concavo-convex |
| 1. Primary (Terminal) | | |
| Capitulum | | |
| Number per primary shoot | 1 | 1 |
| Circumference: | 32.385-38.1 cm (12¾-15 inches) | 32.385-38.1 cm (12¾-15 inches) |
| Weight: | Average 425 grams (0.94 lbs.) | Average 425 grams (0.94 lbs.) |
| Height: | Average 10.16 cm (4 inches) | Average 10.16 cm (4 inches) |
| Diameter: | 11.43-12.7 cm (4½-5 inches) | 11.43-12.7 cm (4½-5 inches) |
| 2. Secondary capitulum | | |
| Number per primary shoot | 10-19 | 3-5 |
| Circumference | 12.065-34.29 cm (4 ¾-13½ | 7.62-38.1 cm (3-15 inches) |
| | inches) | |
| Weight: | Average 152 grams (0.3351 lbs.) | Average 237 grams (0.52 lbs.) |
| Height: | Average 7.62 cm (3 inches) | Average 8.255 cm (3¼ inches) |
| Diameter: | 2.54-11.43 cm (1-4½ inches) | 2.54-12.7 cm (1-5 inches) |
| 3. Tertiary capitulum | | |
| Number per primary shoot | 10-16 | 2-5 |
| Circumference | 7.62-13.97 cm (3-5½ inches) | 7.62-27.31 cm (3-10¾ inches) |
| Weight: | 36 grams (0.07936 lbs.) | Average 44 grams (0.0970 lbs) |
| Height: | Average 5.08 cm (2 inches) | Average 6.78 cm (2⅔ inches) |
| Diameter: | Average 5.08 cm (2 inches) | 5.08-10.795 cm (2-4¼ inches) |
| Receptacle (heart) - when ready | | |
| for harvest and edible | | |
| Shape: | Concavo-convex | Concavo-convex |
| Circumference: | 8.89-25.4 cm (3½-10 inches) | 8.89-25.4 cm (3½-10 inches) |
| Color: | RHS 150D (Green Group) | RHS 150D (Green Group) |
| Texture: | Firm | Firm |
| Size: | Very thick and wide when | Very thick and wide when |
| | compared to other globe varieties | compared to other globe varieties |
| Peduncle | | |
| Shape: | Columnar | Columnar |
| Length: | 2.54-101.6 cm (1-40 inches) | 2.54-76.2 cm (1-30 inches) |
| Diameter: | .635-4.445 cm (¼-1¾ inches) | .635-3.81 cm (¼-1½ inches) |
| Color: | RHS 142A-142B (Green Group) | RHS 142B-142C (Green Group) |
| Strength: | Strong and rigid | Strong and rigid |
| Surface: | Glabrous, slightly pubescent with | Glabrous, slightly pubescent with |
| | shallow vertical ribbing | shallow vertical ribbing |

### Artichoke Plant Variety ‘PAGA 15-1’

In some aspects, the present disclosure relates to a new and distinct artichoke plant variety (Cynara cardunculus L. var. scolymus) which has been denominated as ‘PAGA 15-1’. This new artichoke plant variety exhibits increased bud yield, size and plant vigor compared to ‘Green Globe’, and produces a large primary bud, about 6-13 secondary buds, and about 6-11 tertiary buds per primary reproductive shoot. Up to 8 of the primary and secondary buds are 12-15 inches in circumference. Between 3-4 of the secondary and tertiary buds are 10¾-12½ inches in circumference or larger. The remaining buds are generally between 3-10½ inches in circumference. The plants produce between 1 and 6 reproductive shoots, in addition to the primary reproductive shoot, in the first production cycle. These secondary reproductive shoots produce less marketable sized buds than the primary shoot, although cultural practices such as cutting the primary reproductive shoot from the plant after harvest can materially increase the size and number of buds produced by these secondary shoots.

‘PAGA 15-1’ exhibits the following distinguishing characteristics when grown under normal horticultural practices in Castroville, Calif.:
1. Large bud size;
2. Primary reproductive shoots produce at least 6 secondary buds, and at least 6 tertiary buds;
3. Plants produce an average of 4 reproductive shoots, in addition to the primary reproductive shoot, during the first production cycle;
4. Bracts and receptacles are wide, and thick;
5. Large diameter peduncle; and
6. Early maturity.

The following detailed descriptions set forth the distinctive characteristics of ‘PAGA 15-1’. The data which define these characteristics is based on observations taken in Castroville, Calif. This description is in accordance with UPOV terminology. Color designations, color descriptions, and other phenotypical descriptions may deviate from the stated values and descriptions depending upon variation in environmental, seasonal, climatic, and cultural conditions. ‘PAGA 15-1’ has not been observed under all possible environmental conditions. The botanical description of ‘PAGA 15-1’ was taken from plants that were 5-6 months old. The indicated values represent averages calculated from measurements of several plants. Color references are primarily to the RHS Colour Chart of The Royal Horticultural Society of London (RHS) (2007 edition). Descriptive terminology follows the Plant Identification Terminology, An Illustrated Glossary, 2^{nd} edition by James G. Harris and Melinda Woolf Harris, unless where otherwise defined.

Classification:
Taxonomic name: Cynara cardunculus L. var. scolymus
Common name: Globe Artichoke
Denomination: ‘PAGA 15-1’

Parentage:
Cynara cardunculus var. scolymus L. ‘PAGA 15-1’ was discovered through successive plant selections, and tissue culture propagation cycles of Cynara cardunculus L. var. scolymus variety ‘Green Globe’.

Plant Characteristics Table:

| General information | PAGA 15-1 | Green Globe |
|---|---|---|
| Propagation Method: | Tissue culture | Crown division |
| Months of harvest: | Year round with greatest | Year round with greatest |
| | productivity February through | productivity February through |
| | June | June |
| Number of days from | Range: 165-203 Average: 190 | Range: 161-212 Average 195 |
| planting to first harvest | (tissue culture) | (crown division) |
| Duration of harvest cycle: | Average: 72 days | Average: 232 days |
| Plant | PAGA 15-1 | Green Globe |
| Height range at harvest | 167.64-177.8 cm (66-74 inches) | 119.38-167.64 cm (47-66 inches) |
| (vegetative): | | |
| Height average at harvest | 182.88 cm (72 inches) | 147.32 cm (58 inches) |
| (vegetative): | | |
| Width at harvest (vegetative): | 152.4-284.48 cm (60-112 inches) | 114.3-132.08 cm (45-52 inches) |
| Growth Habit: | Upright, with horizontal spread of | Upright, with horizontal spread of |
| | older leaves and shoots | older leaves and shoots |
| Form: | Arch | Arch |
| Vigor: | Very Good | Moderate |
| Root system: | Thick and fibrous | Thick and fibrous |
| Foliage Density: | Dense, increasing as secondary | Moderately dense. |
| | reproductive shoots develop | |
| Primary Reproductive | | |
| Shoot | PAGA 15-1 | Green Globe |
| Height to primary (terminal) | 152.4 cm-193.04 (60-76 inches) | 106.68-137.16 cm (42-54 inches) |
| bud top: | | |
| Number of leaves (rosette) | 25-32 (average 29) | 14-28 (average 20) |
| Secondary Reproductive | | |
| Shoots | PAGA 15-1 | Green Globe |
| Number per plant in first | 1-6 (average 4) | 1-2 (average 1) |
| production cycle: | | |
| Height to primary (terminal) | 40.64-121.92 cm (16-48 inches) | 13.97-76.2 cm (5.5 to 30 inches) |
| bud top: | | |
| Number of leaves | 7-25 (average 17) | 3-16 (average 7.5) |
| Foliage | PAGA 15-1 | Green Globe |
| Venation: | Reticulate | Reticulate |
| Leaf ratio (L/W): | Approximately 1.1-3.23 | Approximately 1.12-2.28 |
| Upper leaf surface color: | RHS 143A-143B (Green Group) | RHS 141A-141B (Green Group) |
| Lower leaf surface color: | RHS 141D (Green Group) | RHS 136D (Green Group) |
| Leaf texture: | Glabrous and slightly glossy | Glabrous and slightly glossy |
| | appearance in cool, damp | appearance in cool, damp |
| | conditions with interveinal texture. | conditions with interveinal |
| | | texture. |
| Pubescence: | Slightly tomentose on upper | Slightly tomentose on upper |
| | surface, moderately tomentose on | surface, moderately tomentose on |
| | lower. More visible in dry/warm | lower. More visible in dry/warm |
| | conditions. | conditions. |
| Petiole color: | RHS 142B-142C (Green Group) | RHS 142C-142D (Green Group) |
| Petiole and mid rib texture: | Furrowed on upper side with lower | Furrowed on upper side with |
| | side deeply ribbed with ridges | lower side deeply ribbed with |
| | running parallel to the lengthwise | ridges running parallel to the |
| | orientation. Sparsely arachnoid. | lengthwise orientation. Sparsely |
| | | arachnoid. |
| 1. Basal rosette leaves | PAGA 15-1 | Green Globe |
| Shape: | Deeply pinnatifid with sparsely | Deeply pinnatifid with sparsely |
| | ciliate margins, narrowly trullate | ciliate margins, narrowly trullate |
| | overall. Narrowly acuminate apex | overall. Narrowly acuminate |
| | is less acute than Green Globe. | apex. |
| Size: | Very large, >198.12-72.55 cm (78 × | Very large, >172.72 × 45.72 cm |
| | 32.5 inches) | (68 × 18 inches) |
| Distance between leaf | 6.35-12.7 cm (2½-5 inches) | 6.35-11.43 cm (2½-4½ |
| division: | | inches) |
| Division indentation length: | 16.51-41.275 cm (6½-16¼ | 16.51-41.275 cm (6½-16¼ |
| | inches) | inches) |
| Petiole length: | 30.48-57.15 cm (12-22½ inches) | 30.48-48.26 cm (12-19 inches) |
| Petiole width: | 3.175-9.525 cm (1¼-3¾ | 3.175-6.985 cm (1¼-2¾ |
| | inches) | inches) |
| Petiole thickness: | 0.635-1.905 cm (¼-¾ inches) | 0.635-1.905 cm (¼-¾ inches) |
| 2. Cauline leaves | PAGA 15-1 | Green Globe |
| Shape: | Ensiform overall, uneven and | Ensiform overall, uneven and |
| | sparse serrations with narrowly | sparse serrations with narrowly |
| | acuminate apex. Somewhat | acuminate apex. Somewhat |
| | undulate and dente and sparsely | undulate and dente and sparsely |
| | ciliate at margins. | ciliate at margins. |
| Number of leaves: | Approximately 100 | Approximately 73 |
| Size: | 17.78-60.96 cm (7-24 inches) | 17.78-60.96 cm (7-24 inches) |
| Stem (primary reproductive | | |
| stem system) | PAGA 15-1 | Green Globe |
| Length (ground level to base | 152.4 cm-193.04 (60-76 inches) | 106.68-127.0 cm (42-50 inches) |
| of terminal capitulum (bud)): | | |
| Circumference: (base of | 7.62-13.97 cm (3-5½ inches) | 7.62-12.065 cm (3-4.75 inches) |
| terminal capitulum (bud): | | |
| Circumference (base of | Average: 35.56 cm (14 inches) | Average: 24.13 cm (9½ inches) |
| stem): | | |
| Exterior texture: | Vertically ribbed, pubescent | Vertically ribbed, pubescent |
| Stem habit: | Upright, caulescent | Upright, caulescent |
| Pubescence: | Sparsely arachnoid | Sparsely arachnoid |
| Color: | RHS 142A-142B | RHS 142B-142C |
| Involucre bracts | PAGA 15-1 | Green Globe |
| Arrangement: | Imbricate | Imbricate |
| Quantity per Capitulum | 52 outer-35 inner | 52 outer-35 inner |
| (average): | | |
| Outer bract | PAGA 15-1 | Green Globe |
| Shape: | Predominately ovate with flattened | Predominately ovate with |
| | base | flattened base |
| Apex: | Retuse | Retuse |
| Margin: | Entire | Entire |
| Base: | Slightly rounded | Slightly rounded |
| Texture: | Firm, thick and fleshy | Firm, thick and fleshy |
| Color: | RHS 142A-142B (Green Group) | RHS 141C-141D (Green Group) |
| Surface (outer/inner): | Glabrous | Glabrous |
| Spinosity: | Frequent, woody | Moderate frequency, woody |
| Length of spines: | 1-5 mm (0.04-0.2 inches) | 1-5 mm (0.04-0.2 inches) |
| Striation at base of lower | Very minor-only present on the | Present on approximately 20% of |
| bracts: | margins of a few lower outside | lower outside bracts. |
| | bracts. More apparent on immature | |
| | buds. | |
| Striation color: | RHS 77A (Purple-Violet Group) | RHS 77A (Purple-Violet Group) |
| Inner bract | PAGA 15-1 | Green Globe |
| Shape: | Lanceolate with caudate tip | Lanceolate with caudate tip |
| Apex: | Retuse | Retuse |
| Margin: | Entire | Entire |
| Texture: | Firm, thinner than outer bract, | Firm, thinner than outer bract, |
| | fleshy | fleshy |
| Base: | Slightly rounded | Slightly rounded |
| Color: | RHS 150B-150C (Green Group) | RHS 150B-150C (Green Group) |
| Surface (outer/inner): | Glabrous | Glabrous |
| Spinosity: | Finer than outer bract, more | Finer than outer bract more |
| | frequent | frequent |
| Length of spines: | 1-2 mm (0.04-0.079 inches) | 1-2 mm (0.04-0.079 inches) |
| Capitulum (head/bud) - | | |
| when ready for harvest as | | |
| edible bud | PAGA 15-1 | Green Globe |
| Color: | RHS 142A-142B (Green Group) | RHS 141C-141D (Green Group) |
| Shape: | Globose to ovoid | Globose to ovoid |
| Apex: | Broadly rounded to obtuse | Broadly rounded to obtuse |
| Base: | Truncate to rounded | Truncate to rounded |
| Form: | Tightly compact | Tightly compact |
| Texture: | Firm | Firm |
| Receptacle shape: | Concavo-convex | Concavo-convex |
| 1. Primary (Terminal) | | |
| Capitulum | PAGA 15-1 | Green Globe |
| Number per primary shoot | 1 | 1 |
| Circumference: | 34.925-43.18 cm (13¾-17 | 32.385-38.1 cm (12¾-15 |
| | inches) | inches) |
| Weight: | Average 470 grams (1.03 lbs) | Average 425 grams (0.94 lbs.) |
| Height: | Average 10.795 cm (4¼ inches) | Average 10.16 cm (4 inches) |
| Diameter: | 12.7-15.24 cm (5-6 inches) | 11.43-12.7 cm (4½-5 inches) |
| 2. Secondary capitulum | PAGA 15-1 | Green Globe |
| Number per primary shoot | 6-13 | 3-5 |
| Circumference | 7.62-33.02 cm (3-13 inches) | 7.62-38.1 cm (3-15 inches) |
| Weight: | Average 199.3 grams (0.4394 lbs.) | Average 237 grams (0.52 lbs.) |
| Height: | Average 8.255 cm (3¼ inches) | Average 8.255 cm (3¼ inches) |
| Diameter: | 2.54-10.795 cm (1-4¼ inches) | 2.54-12.7 cm (1-5 inches) |
| 3. Tertiary capitulum | PAGA 15-1 | Green Globe |
| Number per primary shoot | 6-11 | 2-5 |
| Circumference | 7.62-15.875 cm (3-6¼ inches) | 7.62-27.31 cm (3-10¾ inches) |
| Weight: | Average 36 grams (0.07936 lbs.) | Average 44 grams (0.0970 lbs) |
| Height: | Average 7.62 cm (3 inches) | Average 6.78 cm (2⅔ inches) |
| Diameter: | 5.08-8.255 cm (2-3¼ inches) | 5.08-10.795 cm (2-4¼ inches) |
| Receptacle (heart) - when | | |
| ready for harvest and edible | PAGA G-1 | Green Globe |
| Shape: | Concavo-convex | Concavo-convex |
| Circumference: | 8.89-25.4 cm (3½-10 inches) | 8.89-25.4 cm (3½-10 inches) |
| Color: | RHS 150D (Green Group) | RHS 150D (Green Group) |
| Texture: | Firm | Firm |
| Size: | Very thick and wide when | Very thick and wide when |
| | compared to other globe varieties | compared to other globe |
| | | varieties |
| Peduncle | PAGA 15-1 | Green Globe |
| Shape: | Columnar | Columnar |
| Length: | 2.54-101.6 cm (1-40 inches) | 2.54-76.2 cm (1-30 inches) |
| Diameter: | .635-4.445 cm (¼-1¾ inches) | .635-3.81 cm (¼-1½ |
| | | inches) |
| Color: | RHS 142A-142B (Green Group) | RHS 142B-142C (Green |
| | | Group) |
| Strength: | Strong and rigid | Strong and rigid |
| Surface: | Glabrous, slightly pubescent with | Glabrous, slightly pubescent |
| | shallow vertical ribbing | with shallow vertical ribbing |

Accordingly, in some embodiments, the present invention is directed to an artichoke plant variety designated ‘PAGA G-1’, wherein a representative sample of tissue culture of said variety has been deposited under DSMZ Accession Number X1. In some embodiments, the present invention is directed to an artichoke plant variety designated ‘PAGA 13-1’, wherein a representative sample of tissue culture of said variety has been deposited under DSMZ Accession Number X2. In some embodiments, the present invention is directed to an artichoke plant variety designated ‘PAGA 15-1’, wherein a representative sample of tissue culture of said variety has been deposited under DSMZ Accession Number X3.

### Tissue Culture

Regeneration of the artichoke plant varieties of the present invention may be achieved by tissue culture. As used herein, the term “tissue culture” indicates a composition containing isolated cells of the same or a different type or a collection of such cells organized into parts of a plant, as well as the process of obtaining thereof. Exemplary types of tissue cultures are protoplasts, calli, meristematic cells, and plant cells that can generate tissue culture that are intact in plants or parts of plants, such as leaves, pollen, embryos, roots, root tips, anthers, pistils, flowers, seeds, petioles, and the like. Techniques of tissue culture of artichoke and regeneration of plants therefrom are known in the art. For example, reference may be made to Murashige, Annual review of plant physiology 25.1 (1974): 135-166, and Bedini et al., Open Life Sciences 7.4 (2012): 680-689.

In some embodiments, the present invention is directed to an artichoke plant regenerated from the tissue culture of ‘PAGA G-1’, ‘PAGA 13-1’, or ‘PAGA 15-1’, wherein said plant expressing all the morphological and physiological characteristics of ‘PAGA G-1’, ‘PAGA 13-1’, or ‘PAGA 15-1’.

In some embodiments, the present invention is directed to a plant part from the plant of ‘PAGA G-1’, ‘PAGA 13-1’, or ‘PAGA 15-1’. In some embodiments, the plant part is a capitulum, a flower, a leaf, a stem, a seed, a cell, a tissue culture, or a portion thereof.

### Breeding

Pedigree breeding and recurrent selection breeding methods may be used to develop artichoke plant varieties from breeding populations. Breeding programs may be used to combine desirable traits from two or more varieties or various broad-based sources into breeding pools from which new varieties are developed by selfing and selection of desired phenotypes. The new varieties are crossed with other varieties and the hybrids from these crosses are evaluated to determine which have commercial potential.

Pedigree breeding may be used for the improvement of self-pollinating crops or inbred lines of cross-pollinating crops. Two parents which possess favorable, complementary traits are crossed to produce an F₁. An F₂ population is produced by selfing one or several F₁'s or by intercrossing two F₁'s (sib mating). Selection of the best individuals is usually begun in the F₂ population. Then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new varieties.

Mass and recurrent selections may be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding may be used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

In some embodiments, the present invention is directed to a method of producing an artichoke plant, comprising crossing the plant of ‘PAGA G-1’, ‘PAGA 13-1’, or ‘PAGA 15-1’ with a second artichoke plant one or more times, and selecting progeny from said crossing.

In some embodiments, the present invention is directed to a method of producing an artichoke plant, comprising selfing the plant of ‘PAGA G-1’, ‘PAGA 13-1’, or ‘PAGA 15-1’ one or more times, and selecting progeny from said selfing.

In some embodiments, the present invention is directed to progeny of the artichoke plant variety ‘PAGA G-1’, ‘PAGA 13-1’, or ‘PAGA 15-1’, wherein said progeny retain all the physiological and morphological characteristics of variety ‘PAGA G-1’, ‘PAGA 13-1’, or ‘PAGA 15-1’ when grown under the same environmental conditions.

In some embodiments, the present invention is directed to an essentially derived variety (EDV) of the artichoke plant variety ‘PAGA G-1’, ‘PAGA 13-1’, or ‘PAGA 15-1’ having one, two or three physiological and/or morphological characteristics that are different from those of the plant of ‘PAGA G-1’, ‘PAGA 13-1’, or ‘PAGA 15-1’, but otherwise having all the physiological and morphological characteristics of the artichoke plant variety ‘PAGA G-1’, ‘PAGA 13-1’, or ‘PAGA 15-1’. The term “essentially derived” is used herein as defined in the International Convention for the Protection of New Varieties of Plants of Dec. 2, 1961, as revised on Mar. 19, 1991 (UPOV), according to which (Chap. V, Art. 14, par. (5)(b)) a variety is “essentially derived” from another variety (“the initial variety”) when: (i) it is predominantly derived from the initial variety, or from a variety that is itself predominantly derived from the initial variety, while retaining the expression of the essential characteristics that result from the genotype or combination of genotypes of the initial variety; (ii) it is clearly distinguishable from the initial variety; and (iii) except for the differences which result from the act of derivation, it conforms to the initial variety in the expression of the essential characteristics that result from the genotype or combination of genotypes of the initial variety. Such essentially-derived varieties may be obtained for example by the selection of a natural or induced mutant, or of a somaclonal variant, the selection of a variant individual from plants of the initial variety, backcrossing, or transformation by genetic engineering (see UPOV, Chap. V, Art. 14, par. (5)(c)).

### Propagation

In some embodiments, the present invention is directed to a method of producing an artichoke plant, or a part thereof, of the artichoke plant variety ‘PAGA G-1’, ‘PAGA 13-1’, or ‘PAGA 15-1’, comprising vegetative propagation of the plant of ‘PAGA G-1’, ‘PAGA 13-1’, or ‘PAGA 15-1’. As used herein, the terms “propagation” and “reproduction” are used interchangeably to refer to the process of a progeny plant being generated from a plant part of a parent plant. There are two main types of propagation in plants: sexual propagation and asexual propagation. The term “sexual propagation” refers to generating a new plant from a seed. The term “asexual propagation”, “vegetative propagation” or “clonal propagation” refers to generating a new plant from a part of a plant of a parent plant that is not a seed. An artichoke plant may be propagated by sexual propagation or asexual propagation. The term “propagation material” or “propagating material” refers to a plant part that is used to propagate plants. For sexual propagation, the propagating material is a seed. For asexual propagation, the propagating material may be any non-seed plant part that is capable of regenerating into a new plant. For a more detailed description of plant propagation, see Hartmann and Kester (1975) Plant propagation: principles and practices (No. SB 119 H3 1975).

Accordingly, in some embodiments, the vegetative propagation comprises regenerating a whole artichoke plant from a part of ‘PAGA G-1’, ‘PAGA 13-1’, or ‘PAGA 15-1’. In some embodiments, the part is a cutting, a cell culture, a tissue culture, a stump, a sucker, a shoot, an offshoot or an ovoli. In some embodiments, the regenerated whole artichoke plant has all the morphological and physiological characteristics of ‘PAGA G-1’, ‘PAGA 13-1’, or ‘PAGA 15-1’ when grown under the same environmental conditions. In some embodiments, the present invention is directed to a plant part of the regenerated whole artichoke plant, wherein said plant part is a harvested capitulum, a pollen grain, a cell, a leaf, a petiole, a shoot, a stem, a root, a cutting, a stump, an offshoot, an ovoli, a receptacle, a bract, a flower, a floret, a flower bud, a seed, or a portion thereof. In some embodiments, the present invention is directed to a food or feed product comprising said plant part. In some embodiments, the plant part is fresh. In some embodiments, the plant part is processed.

### Transformation

As used herein, the term “transformation” and “transforming” a plant cell encompasses all techniques by which a nucleic acid molecule may be introduced into such a cell. Examples include, but are not limited to, transfection with viral vectors; transformation with plasmid vectors; electroporation; microinjection; Agrobacterium-mediated transfer; direct DNA uptake; Whiskers-mediated transformation; and microprojectile bombardment. Methods for transforming plant cells, plants and portions thereof are described in Draper et al., 1988, Plant Genetic Transformation and Gene Expression. A Laboratory Manual, Blackwell Sci. Pub. Oxford, p. 365; Potrykus and Spangenburg, 1995, Gene Transfer to Plants. Springer-Verlag, Berlin; and Gelvin et al., 1993, Plant Molecular Biol. Manual. Kluwer Acad. Pub. Dordrecht. A review of transgenic plants, including transformation techniques, is provided in Galun and Breiman, 1997, Transgenic Plants. Imperial College Press, London.

Accordingly, in some embodiments, the present invention is directed to a method of producing an artichoke plant having a desired trait, wherein the method comprises transforming the artichoke plant of ‘PAGA G-1’, ‘PAGA 13-1’, or ‘PAGA 15-1’ with a transgene that confers the desired trait, wherein the transformed plant retains all the phenotypic and morphological characteristics of variety ‘PAGA G-1’, ‘PAGA 13-1’, or ‘PAGA 15-1’ and contains the desired trait.

### Genotyping

As used herein, the term “genotype” refers to the genetic makeup of a cell, an organism, or an individual in a population and is usually with reference to a specific character or phenotypic trait of interest under consideration. As used herein, the term “genotyping” or “determining the genotype” refers to the process of determining genetic variations (polymorphisms) among individuals in a species. There are many laboratory-based techniques known in the art that are available for genotyping. Examples include, without limitation, Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs) which are also referred to as Microsatellites, and Single Nucleotide Polymorphisms (SNPs).

Accordingly, in some embodiments, the present invention is directed to a method of determining the genotype of a plant of ‘PAGA G-1’, ‘PAGA 13-1’, or ‘PAGA 15-1’, comprising obtaining a sample of nucleic acids from said plant and detecting in said nucleic acids a plurality of polymorphisms.

### DEPOSIT INFORMATION

### Artichoke Plant Variety ‘PAGA G-1’

A deposit of the plant tissue cultures for artichoke plant variety ‘PAGA G-1’ disclosed in this invention is maintained by Luis A Scattini & Sons LP, having an address at 55 East San Joaquin Street, Salinas, Calif. 93901, United States. Access to this deposit will be available during the pendency of this application to persons determined by the Commissioner of Patents and Trademarks to be entitled thereto under 37 C.F.R. § 1.14 and 35 U.S.C. § 122. Upon allowance of any claims in this application, all restrictions on the availability to the public of the deposit will be irrevocably removed by affording access to a deposit of at least 18 cryopreserved vials of plant tissue cultures made according to the Budapest Treaty in the Leibniz Institute DSMZ-German Collection of Microorganisms and Cell Cultures (DSMZ), Inhoffenstr. 7 B, D-38124 Braunschweig, Germany.

At least 18 cryopreserved vials of the plant tissue cultures disclosed in the invention were deposited on DATE1 according to the Budapest Treaty in the Leibniz Institute DSMZ-German Collection of Microorganisms and Cell Cultures (DSMZ), Inhoffenstr. 7 B, D-38124 Braunschweig, Germany. The deposit has been assigned DSMZ number X1. Access to this deposit will be available during the pendency of this application to persons determined by the Commissioner of Patents and Trademarks to be entitled thereto under 37 C.F.R. § 1.14 and 35 U.S.C. § 122. Upon allowance of any claims in this application, all restrictions on the availability to the public of the deposit will be irrevocably removed.

The deposit will be maintained in the DSMZ depository, which is a public depository, for a period of at least 30 years, or at least 5 years after the most recent request for a sample of the deposit, or for the effective life of the patent, whichever is longer, and will be replaced if a deposit becomes nonviable during that period.

### Artichoke Plant Variety ‘PAGA 13-1’

A deposit of the plant tissue cultures for artichoke plant variety ‘PAGA 13-1’ disclosed in this invention is maintained by Luis A Scattini & Sons LP, having an address at 55 East San Joaquin Street, Salinas, Calif. 93901, United States. Access to this deposit will be available during the pendency of this application to persons determined by the Commissioner of Patents and Trademarks to be entitled thereto under 37 C.F.R. § 1.14 and 35 U.S.C. § 122. Upon allowance of any claims in this application, all restrictions on the availability to the public of the deposit will be irrevocably removed by affording access to a deposit of at least 18 cryopreserved vials of plant tissue cultures made according to the Budapest Treaty in the Leibniz Institute DSMZ-German Collection of Microorganisms and Cell Cultures (DSMZ), Inhoffenstr. 7 B, D-38124 Braunschweig, Germany.

At least 18 cryopreserved vials of the plant tissue cultures disclosed in the invention were deposited on DATE2 according to the Budapest Treaty in the Leibniz Institute DSMZ-German Collection of Microorganisms and Cell Cultures (DSMZ), Inhoffenstr. 7 B, D-38124 Braunschweig, Germany. The deposit has been assigned DSMZ number X2. Access to this deposit will be available during the pendency of this application to persons determined by the Commissioner of Patents and Trademarks to be entitled thereto under 37 C.F.R. § 1.14 and 35 U.S.C. § 122. Upon allowance of any claims in this application, all restrictions on the availability to the public of the deposit will be irrevocably removed.

The deposit will be maintained in the DSMZ depository, which is a public depository, for a period of at least 30 years, or at least 5 years after the most recent request for a sample of the deposit, or for the effective life of the patent, whichever is longer, and will be replaced if a deposit becomes nonviable during that period.

### Artichoke Plant Variety ‘PAGA 15-1’

A deposit of the plant tissue cultures for artichoke plant variety ‘PAGA 15-1’ disclosed in this invention is maintained by Luis A Scattini & Sons LP, having an address at 55 East San Joaquin Street, Salinas, Calif. 93901, United States. Access to this deposit will be available during the pendency of this application to persons determined by the Commissioner of Patents and Trademarks to be entitled thereto under 37 C.F.R. § 1.14 and 35 U.S.C. § 122. Upon allowance of any claims in this application, all restrictions on the availability to the public of the deposit will be irrevocably removed by affording access to a deposit of at least 18 cryopreserved vials of plant tissue cultures made according to the Budapest Treaty in the Leibniz Institute DSMZ-German Collection of Microorganisms and Cell Cultures (DSMZ), Inhoffenstr. 7 B, D-38124 Braunschweig, Germany.

At least 18 cryopreserved vials of the plant tissue cultures disclosed in the invention were deposited on DATE3 according to the Budapest Treaty in the Leibniz Institute DSMZ-German Collection of Microorganisms and Cell Cultures (DSMZ), Inhoffenstr. 7 B, D-38124 Braunschweig, Germany. The deposit has been assigned DSMZ number X3. Access to this deposit will be available during the pendency of this application to persons determined by the Commissioner of Patents and Trademarks to be entitled thereto under 37 C.F.R. § 1.14 and 35 U.S.C. § 122. Upon allowance of any claims in this application, all restrictions on the availability to the public of the deposit will be irrevocably removed.

The deposit will be maintained in the DSMZ depository, which is a public depository, for a period of at least 30 years, or at least 5 years after the most recent request for a sample of the deposit, or for the effective life of the patent, whichever is longer, and will be replaced if a deposit becomes nonviable during that period.

We claim:

1. An artichoke plant variety selected from the group consisting of: 1) an artichoke plant variety designated ‘PAGA G-1’, wherein a representative sample of tissue culture of said variety has been deposited under DSMZ Accession Number X1; 2) an artichoke plant variety designated ‘PAGA 13-1’, wherein a representative sample of tissue culture of said variety has been deposited under DSMZ Accession Number X2; and 3) an artichoke plant variety designated ‘PAGA 15-1’, wherein a representative sample of tissue culture of said variety has been deposited under DSMZ Accession Number X3.

2. An artichoke plant regenerated from the tissue culture of the artichoke plant variety of claim 1, wherein said plant expressing all the morphological and physiological characteristics of the artichoke plant variety of claim 1.

3. A plant part from the artichoke plant variety of claim 1.

4. The plant part of claim 3, wherein said part is a capitulum, a flower, a leaf, a stem, a seed, a cell, a tissue culture, or a portion thereof.

5. A method of producing an artichoke plant, comprising crossing a plant of the artichoke plant variety of claim 1 with a second artichoke plant one or more times, and selecting progeny from said crossing.

6. A method of producing an artichoke plant, comprising selfing the plant of the artichoke plant variety of claim 1 one or more times, and selecting progeny from said selfing.

7. Progeny of the artichoke plant variety of claim 1, wherein said progeny retain all the physiological and morphological characteristics of the artichoke plant variety of claim 1 when grown under the same environmental conditions.

8. An essentially derived variety (EDV) of the artichoke plant variety of claim 1 having one, two or three physiological and/or morphological characteristics that are different from those of the artichoke plant variety of claim 1, but otherwise having all the physiological and morphological characteristics of the artichoke plant variety of claim 1.

9. A method of producing an artichoke plant, or a part thereof, of the artichoke plant variety of claim 1, comprising vegetative propagation of the plant of the artichoke plant variety of claim 1.

10. The method of claim 9, wherein said vegetative propagation comprises regenerating a whole artichoke plant from a part of the artichoke plant variety of claim 1.

11. The method of claim 10, wherein said part is a cutting, a cell culture, a tissue culture, a stump, a sucker, a shoot, an offshoot, or an ovoli.

12. The method of claim 10, wherein the regenerated whole artichoke plant has all the morphological and physiological characteristics of the artichoke plant variety of claim 1 when grown under the same environmental conditions.

13. A plant part of the regenerated whole artichoke plant of claim 12, wherein said plant part is a harvested capitulum, a pollen grain, a cell, a leaf, a petiole, a shoot, a stem, a root, a cutting, a stump, an offshoot, an ovoli, a receptacle, a bract, a flower, a floret, a flower bud, a seed, or a portion thereof.

14. The plant part of claim 13, wherein said plant part is a seed.

15. A food or feed product comprising a plant part of claim 14.

16. An artichoke plant produced by growing the seed of claim 14.

17. The food or feed product of claim 15, wherein said plant part is fresh or processed.

18. A method of producing an artichoke plant having a desired trait, wherein the method comprises transforming a plant of the artichoke plant variety of claim 1 with a transgene that confers the desired trait, wherein the transformed plant retains all the phenotypic and morphological characteristics of the artichoke plant variety of claim 1 and contains the desired trait.

19. An artichoke plant produced by the method of claim 18, wherein the plant comprises the desired trait and all the physiological and morphological characteristics of the artichoke plant variety of claim 1.

20. A method of determining the genotype of a plant of the artichoke plant variety of claim 1 comprising obtaining a sample of nucleic acids from said plant and detecting in said nucleic acids a plurality of polymorphisms.
